(19) United States
(12) Patent Application Publication
(10) Pub. No.: US 2021/0324425 A1
(43) Pub. Date: Oct. 21, 2021

(54) CELL-FREE METABOLIC PATHWAY OPTIMIZATION THROUGH REMOVAL OF SELECT PROTEINS

(71) Applicant: UT-Battelle, LLC, Oak Ridge, TN (US)

(72) Inventors: Mitchel J. Doktycz, Oak Ridge, TN (US); Jaime Lorenzo N Dinglasan, Oak Ridge, TN (US); David Garcia, Oak Ridge, TN (US); Ben P. Mohr, Oak Ridge, TN (US)

(21) Appl. No.: 17/235,450

(22) Filed: Apr. 20, 2021

Related U.S. Application Data
(60) Provisional application No. 63/013,066, filed on Apr. 21, 2020.

(51) Int. Cl. C12P 7/22; C12N 1/06; C12P 7/06

(52) U.S. Cl. CPC C12P 7/22; C12P 7/06; C12N 1/06

(57) ABSTRACT

The present disclosure is directed to methods for proteome engineering cells such that cell-free extracts prepared from such engineered cells can be modified to have metabolic flux directed to a metabolism of interest. In addition, methods for producing cell-free extracts with directed metabolism, cell-free extracts and kits that contain cell-free extracts are also disclosed.

### CROSS REFERENCE TO RELATED APPLICATION

This application claims the benefit of priority from U.S. Provisional Application No. 63/013,066, filed Apr. 21, 2020, the entire contents of which are incorporated herein by reference.

### INCORPORATION BY REFERENCE OF SEQUENCE LISTING

The Sequence Listing in an ASCII text file, named as 39345_4430_1_SequenceListing.txt of 3 KB, created on Apr. 16, 2021, and submitted to the United States Patent and Trademark Office via EFS-Web, is incorporated herein by reference.

### STATEMENT REGARDING FEDERALLY SPONSORED RESEARCH OR DEVELOPMENT

This invention was made with government support under Prime Contract No. DE-AC05-000R22725 awarded by the U.S. Department of Energy. The government has certain rights in the invention.

### BACKGROUND

The use of cell-free extracts for metabolite production has been significantly studied and several prominent labs have shown its efficacy as a potential production platform. However, as more work has been undertaken, it has been shown that cell-free extracts are not without inefficiencies. For instance, cell-free extracts fed with glucose while capable of consuming the substrate will disperse it to deleterious metabolic pathways.

Driven by the prospect of biological systems that can be easily manipulated, the application of synthetic biology tools to in vitro environments offers a promising approach to harnessing an organism's rich metabolic potential. Cell-free systems use cytoplasmic components, devoid of genetic material and membranes, as a means of producing complex chemical transformations. While living cells require membranes, growth substrates, and biochemical regulation, in vitro systems sidestep these barriers to manipulation and present an opportunity to explicitly define a system for creating novel proteins and metabolites. In this way, cell-free metabolic engineering (CFME) can use the organism's existing biochemical functions and further combine these capabilities with heterologous pathways to produce chemical precursors, biofuels, and pharmaceuticals.

Efforts to engineer cell-free systems have taken different approaches. Ideally, a CFME system would contain a minimal set of components necessary to carry out a desired biochemical process. Previous approaches employed a defined set of purified enzymes for producing high-yielding chemical conversions and have successfully demonstrated a variety of capabilities including chemical production and protein synthesis. Constructing complex, multistep pathways require significant development and upfront costs as utilizing purified proteins at scale remains costly. Further, these purified component systems can exhibit slow catalysis rates, possibly due to the lack of accessory proteins and appropriate protein concentrations capable of improving pathway yield. Nevertheless, long-running CFME systems that can catalyze multi-step reaction pathways for days have been developed.

The use of crude cell extracts presents an alternative approach to CFME. Simple cell lysis and minimal fractionation can be rapidly carried out and result in complex enzyme mixtures for a fraction of the cost of purified components. Crude extract systems derived from both commonly used cell-free model organisms, such as E. coli BL21 Star (DE3), or nontraditional strains, such as Vibrio natriegens, contain a similar biochemistry to the donor cell and can serve as both prototyping tools for in vivo metabolic engineering and as bioproduction platforms. Cell-free systems work well for both prototyping and production as CFME can be modularly assembled with lysates enriched for specific enzymes or entire metabolic pathways in order to produce a specific molecule. Additionally, their compatibility with chemical reactors and ability to consume low-cost feedstocks have popularized them as potential sources for industrial production. These combined capabilities allow CFME processes to make use of tools from traditional bioproduction platforms while taking advantage of the open and modular nature of cell-free systems.

While environmental variables of a cell-free system can be easily manipulated, the proteomic content of the crude extract is more difficult to engineer. Genetic manipulation of a donor strain can substantially impact its growth and function as a bioproduction system. It has been noted previously that simple variations in growth conditions can lead to complex changes in the proteome and significant differences in metabolite flux in the resulting crude extracts. Further, specific enzymes can be added or expressed in an extract to further define metabolite production. However, removing specific proteins is challenging as gene deletions can affect the growth and global expression of the donor cell. In particular, deletions to central metabolism can be lethal, which severely limits the ability to direct flux from simple carbon sources. The inability to remove specific pathways from CFME reactions poses a significant constraint and limits the use of crude extracts for bioproduction. Tools that allow shaping of the cell-free proteome have been proposed but have not been applied towards the manipulation of cell-free metabolism. Instead, these efforts have focused on improving various single aspects of transcription and translation. Providing approaches with the ability to modulate the presence of multiple enzymes and specific pathways will be critical in enabling the use of crude extract systems for metabolic engineering applications.

### SUMMARY OF THE DISCLOSURE

An aspect of this disclosure is directed to a method of genetic engineering a cell so that the cell-free extract made from the genetically engineered cell can be manipulated to direct metabolic flux to a metabolite of interest.

In some embodiments, the method comprises linking an affinity tag to at least one enzyme in the cell that affects the amount of a metabolite of interest. In some embodiments, the method comprises linking the affinity tag to multiple or all enzymes that affect the amount of the metabolite.

In some embodiments, deletion or inactivation of the at least one enzyme significantly impairs the cell's metabolism or kills the cell.

In some embodiments, the method further comprises expressing in the cell a nucleic acid encoding an exogenous enzyme that affects the concentration of the metabolite. In some embodiments, the exogenous enzyme is an enzyme not native to the cell or an engineered version of a native enzyme.

In some embodiments, the linking of the affinity tag is achieved by a method selected from the group consisting of multiplex automated genome engineering (MAGE), CRISPR/Cas system, Cre/Lox system, TALEN system, ZFNs system and homologous recombination.

In some embodiments, the at least one enzyme is selected from an enzyme in the glycolysis pathway, an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, or an enzyme in the fatty acid metabolism pathway.

In some embodiments, the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, or a metabolite in the fatty acid metabolism pathway.

In some embodiments, the metabolite is selected from pyruvate, ethanol, mevalonate, isopentyl pyrophosphate, or acetyl coenzyme A.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase, or prenyl transferase.

In some embodiments, the metabolite is acetyl coenzyme A, and wherein the enzyme is pyruvate dehydrogenase.

In some embodiments, the cell is a single-cell organism. In some embodiments, the single-cell organism is selected from the genera Lactobacillus, Escherichia, Bacillus, Vibrio, Bifidobacterium, Saccharomyces, Pichia, Pseudomonas, Streptomyces, or Streptococcus.

In some embodiments, the genetically engineered cell is a eukaryotic cell, a prokaryotic cell, or an archaeal cell.

In some embodiments, the affinity tag is selected from a His tag, a FLAG tag, a Strep II tag, a glutathione S-transferase (GST) tag, a Calmodulin binding protein (CBP) tag, a covalent yet dissociable NorpD peptide (CYD) tag, a polyarginine (Poly-Arg or nArg) tag, or a heavy chain of protein C (HPC) tag.

In some embodiments, the genetically engineered cell is a bacterium from genus Escherichia, the metabolite is pyruvate and the at least one enzyme is selected from PpsA, PflB, AceE or LdhA. In some embodiments, each of PpsA, PflB, AceE and LdhA is linked to the affinity tag.

Another aspect of the disclosure is directed to a method for making a cell-free extract that has a directed metabolic flux towards a metabolite of interest comprising: growing a genetically engineered cell under conditions that allow production of the metabolite, wherein at least one enzyme in the genetically engineered cell that affects the amount of metabolite has been engineered to be linked to an affinity tag; making a crude cell extract from the genetically engineered cell; removing the at least one enzyme from the crude cell extract using affinity purification, thereby obtaining a cell-free extract capable of producing the metabolite.

In some embodiments, multiple or all enzymes that affect the amount of the metabolite have been engineered to be linked to an affinity tag and have been substantially removed from the cell extract.

In some embodiments, the at least one enzyme is a central metabolism enzyme and deletion or inactivation of the at least one enzyme significantly impairs the cell's metabolism or kills the cell.

In some embodiments, the genetically engineered cell further comprises a nucleic acid encoding an exogenous enzyme that affects the concentration of the metabolite.

In some embodiments, the exogenous enzyme is selected from an enzyme not native to the cell or an engineered version of a native enzyme.

In some embodiments, the at least one enzyme is selected from an enzyme in the glycolysis pathway, an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, or an enzyme in the fatty acid metabolism pathway.

In some embodiments, the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, or a metabolite in the fatty acid metabolism pathway.

In some embodiments, the metabolite is selected from pyruvate, ethanol, mevalonate, isopentyl pyrophosphate, or acetyl coenzyme A.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase, or prenyl transferase.

In some embodiments, the metabolite is acetyl coenzyme A, and wherein the enzyme is pyruvate dehydrogenase.

In some embodiments, the genetically engineered cell is a single-cell organism, the metabolite is an aromatic compound, and the organism is grown under conditions lacking aromatic amino acids.

In some embodiments, the single-cell organism is selected from the genera Lactobacillus, Escherichia, Bacillus, Vibrio, Bifidobacterium, Saccharomyces, Pichia, Pseudomonas, Streptomyces, or Streptococcus.

In some embodiments, the genetically engineered cell has been cultured in a controlled growth medium before extract preparation. In some embodiments, the controlled growth medium lacks aromatic amino acids or comprises an organic hydrocarbon. In some embodiments, the controlled growth medium comprises a pre-defined temperature, pH, or oxygenation level.

In some embodiments, the genetically engineered cell is a eukaryotic cell, a prokaryotic cell, or an archaeal cell.

In some embodiments, the affinity tag is selected from a His tag, a FLAG tag, a Strep II tag, a glutathione S-transferase (GST) tag, a Calmodulin binding protein (CBP) tag, a covalent yet dissociable NorpD peptide (CYD) tag, a polyarginine (Poly-Arg or nArg) tag, or a heavy chain of protein C tag.

In some embodiments, the genetically engineered cell is a bacterium from genus Escherichia, the metabolite is pyruvate, and the at least one enzyme is selected from PpsA, PflB, AceE or LdhA.

In some embodiments, each of PpsA, PflB, AceE and LdhA is linked to the same affinity tag.

Another aspect of the disclosure is directed to a cell-free extract that has a directed metabolic flux towards a metabolite of interest comprising an extract from a genetically engineered cell, wherein at least one enzyme that affects the amount of the metabolite has been substantially removed from the cell extract. In some embodiments, multiple or all enzymes that affect the amount of the specific metabolite have been substantially removed from the cell extract.

In some embodiments, the at least one enzyme is a central metabolism enzyme that, deletion or inactivation of the at least one enzyme significantly impairs the cell's metabolism or kills the cell.

In some embodiments, the genetically engineered cell further comprises a nucleic acid encoding an exogenous enzyme that affects the concentration of the metabolite.

In some embodiments, the exogenous enzyme is selected from an enzyme not native to the cell or an engineered version of a native enzyme.

In some embodiments, the at least one enzyme is selected from an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, or an enzyme in the fatty acid metabolism pathway.

In some embodiments, the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, or a metabolite in the fatty acid metabolism pathway.

In some embodiments, the metabolite is selected from pyruvate, ethanol, mevalonate, isopentyl pyrophosphate, or acetyl coenzyme A.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase, or prenyl transferase.

In some embodiments, the metabolite is acetyl coenzyme A, and wherein the enzyme is pyruvate dehydrogenase.

In some embodiments, the genetically engineered cell has been engineered such that the at least one enzyme is linked to an affinity tag.

In some embodiments, the affinity tag is selected from a His tag, a FLAG tag, a Strep II tag, a glutathione S-transferase (GST) tag, a Calmodulin binding protein (CBP) tag, a covalent yet dissociable NorpD peptide (CYD) tag, a polyarginine (Poly-Arg or nArg) tag, or a heavy chain of protein C (HPC) tag.

In some embodiments, the genetically engineered cell has been cultured in a controlled growth medium before extract preparation.

In some embodiments, the controlled growth medium lacks aromatic amino acids or comprises an organic hydrocarbon.

In some embodiments, the controlled growth medium comprises a pre-defined temperature, pH, or oxygenation level.

In some embodiments, the genetically engineered cell is a eukaryotic cell, a prokaryotic cell, or an archaeal cell.

In some embodiments, the genetically engineered cell is a single-cell organism.

In some embodiments, the single-cell organism is selected from the genera Lactobacillus, Escherichia, Bacillus, Vibrio, Bifidobacterium, Saccharomyces, Pichia, Pseudomonas, Streptomyces, or Streptococcus.

In some embodiments, the genetically engineered cell is a bacterium from genus Escherichia, the metabolite is pyruvate, and the at least one enzyme is selected from PpsA, PflB, AceE or LdhA. In some embodiments, each of PpsA, PflB, AceE and LdhA is linked to the same affinity tag.

Another aspect of the disclosure is directed to a cell-free extract that has a directed metabolic flux towards a metabolite of interest comprising a reduced extract from a genetically engineered cell, wherein at least one enzyme that affects the amount of the metabolite has been substantially removed from the cell extract. In some embodiments, multiple or all enzymes that affect the amount of the specific metabolite have been substantially removed from the cell extract.

In some embodiments, the at least one enzyme is a central metabolism enzyme that, deletion or inactivation of the at least one enzyme significantly impairs the cell's metabolism or kills the cell.

In some embodiments, the genetically engineered cell further comprises a nucleic acid encoding an exogenous enzyme that affects the concentration of the metabolite.

In some embodiments, the exogenous enzyme is selected from an enzyme not native to the cell or an engineered version of a native enzyme.

In some embodiments, the at least one enzyme is selected from an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, or an enzyme in the fatty acid metabolism pathway.

In some embodiments, the specific metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, or a metabolite in the fatty acid metabolism pathway.

In some embodiments, the metabolite is selected from pyruvate, ethanol, mevalonate, isopentyl pyrophosphate, or acetyl coenzyme A.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase, or prenyl transferase.

In some embodiments, the metabolite is acetyl coenzyme A, and wherein the enzyme is pyruvate dehydrogenase.

In some embodiments, the genetically engineered cell has been engineered such that the at least one enzyme is linked to an affinity tag. In some embodiments, the affinity tag is selected from a His tag, a FLAG tag, a Strep II tag, a glutathione S-transferase (GST) tag, a Calmodulin binding protein (CBP) tag, a covalent yet dissociable NorpD peptide (CYD) tag, a polyarginine (Poly-Arg or nArg) tag, or a heavy chain of protein C (HPC) tag.

In some embodiments, the genetically engineered cell has been cultured in a controlled medium before extract preparation.

In some embodiments, the controlled medium lacks aromatic amino acids or comprises an organic hydrocarbon.

In some embodiments, the controlled medium comprises a pre-defined temperature, pH, or oxygenation level.

In some embodiments, the genetically engineered cell is a eukaryotic cell, a prokaryotic cell, or an archaeal cell.

In some embodiments, the genetically engineered cell is a one-celled organism.

In some embodiments, the one-celled organism is selected from the genera Lactobacillus, Escherichia, Bacillus, Vibrio, Bifidobacterium, Saccharomyces, Pichia, Pseudomonas, Streptomyces, or Streptococcus.

In some embodiments, the genetically engineered cell is a bacterium from genus Escherichia, the specific metabolite is pyruvate, and the at least one enzyme is selected from PpsA, PflB, AceE or LdhA.

In some embodiments, each of PpsA, PflB, AceE and LdhA is linked to the same affinity tag.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Overview of approaches to preparing lysates for cell-free metabolic engineering. (A) Complex metabolism present in E. coli lysates harnessed for cell-free metabolite production can compromise central metabolic precursor yields. (B) & (C) Cell-free metabolic engineering approaches seek to reduce lysate complexity in order to redirect carbon flux and pool central metabolic precursors. (B) The standard CFME approach reduces lysate complexity by deleting target genes from the source strain, often resulting in growth impaired or lethal phenotypes due to the inability to remove essential genes. This can require multiple design-build-test cycles. (C) The new approach involves engineering source strains to endogenously express recognition sequences, such as 6×His-tags, into target proteins for subsequent removal from lysates through affinity purification, resulting in minimal to no impact on source strain growth and enhanced pooling of specific metabolic products.

FIG. 2. Glycolysis and engineered pathway nodes showing the location of the modified enzymes PflB, LdhA, PpsA, and AceE.

FIGS. 3A-3C. Source strain multiplex genome engineering and expected metabolic phenotypes of derived lysates post-depletion. (A) Strain construction course by MAGE cycling culminating with the 6×His-4 containing all 4 tags. Each arrow designates the strain being taken through the MAGE process with the oligos used to transform each strain above the arrow. (B) MASC-PCR results for additive mutations using primers specifically designed for the 6×His-tagged version of the gene. (C) Expected metabolic phenotypes present in WT and engineered lysate proteomes after the depletion of lysates derived from all generated strains.

FIGS. 4A-4F. Relative changes between nondepleted and depleted versions of the lysates in terms of (A) glucose consumption (nondepleted minus depleted), and (B) pyruvate, (C) lactate, (D) ethanol, (E) acetate, and (F) formate production (depleted minus nondepleted) over time. Depleted extracts have had specific 6×His-tagged proteins removed by incubating with them cobalt beads. Extracts containing tagged proteins, but without an incubation step, are referred to as nondepleted. Data for the time course reactions were acquired using n=3 biological replicates. Standard errors calculated for replicates were negligible.

FIGS. 5A-5E. Proteomic analysis of control cell-free extracts without depletion (blue), and a depleted cell-free extract (orange), and the elutions from the depleted extracts (gray). Significant fold-changes in protein concentration when comparing the depleted to the nondepleted extract are denoted by p-value and fold change reduction in concentration of the protein above a bracket. (A) WT, (B) 6×His-pflB, (C) 6×His-2, (D) 6×His-3, and (E) 6×His-4 strains. Asterisks indicate proteins targeted for removal in the depleted strain, each experiment is derived from n=3 biological replicates.

FIG. 6. Simplified metabolic map of phenol biosynthesis by heterologous expression of phenol-tyrosine lyase in E. coli. Only enzymatic transformations found significant in this study are represented. The heterologous enzyme expressed by cell-free protein synthesis is colored red. Symbols: Full yellow circles, ATP; half yellow circles, ADP; empty yellow circles, AMP; full purple circles, NADPH; half purple circles, NADP⁺; full blue circles, NADH; half blue circles, NAD⁺.

FIGS. 7A-7C. (A) Comparison of protein abundance in tyrosine metabolism (including abbreviated glycolysis, pentose phosphate pathway, and shikimate pathway) between complex medium YTPG and defined medium EzGlc. Each box represents the mean log₂(fold change) in protein abundance in a variant growth medium compared to mean protein abundance in the YTPG cell-free system, top and bottom of each box represent the largest and smallest fold change observed for a given protein, error bars represent the 90% confidence interval around the mean. Significance was determined by a two-tailed Student's t-test compared to the YTPG cell-free system: *, p<0.05. Pathway enzymes not depicted can be assumed to have undergone no significant change in abundance. (B) In vitro phenol biosynthesis from ¹³C₆ glucose in a one-pot CFPS-ME reaction measured at 48 hours. Only ¹³C₆ phenol is depicted (m/z=100.1). Values represent averages of technical replicates (n=3) and error bars represent 1 SD. Significance was determined by a two-tailed Student's t-test compared to the YTPG cell-free system: *, p<0.05; ns, p>0.05. (C) Volcano plot of proteomic data. Volcano plots are depicted with the log 2(fold change) in abundance of each protein and the −log 10(p-value) derived from performing a Student's T-test. The average abundance of each protein in the EzGlc cell-free system (n=3) was compared against the average abundance of each protein in the YTPG cell-free system (n=3). Red points show proteins which have been found to be significantly differentially abundant by at least twofold and p<0.01. Black points are not significantly changed.

FIGS. 8A-8B. (A) Comparison of protein abundance in tyrosine metabolism (including abbreviated glycolysis, pentose phosphate pathway, shikimate pathway, arabinose uptake and glycerol uptake) between EzGlc and medium with variant carbon source EzAra and EzGly. Each bar represents the mean log₂(fold change) in protein abundance in a variant growth medium compared to mean protein abundance in the EzGlc cell-free system, top and bottom of each box represent the largest and smallest fold change observed for a given protein, error bars represent the 90% confidence interval around the mean. Significance was determined by a two-tailed Student's t-test compared to the EzGlc cell-free system: *, p<0.05. Pathway enzymes not depicted can be assumed to have undergone no significant change in abundance. (B) In vitro phenol biosynthesis from ¹³C₆ glucose in a one-pot CFPS-ME reaction measured at 48 hours. Only ¹³C₆ phenol is depicted (m/z=100.1). Values represent averages of technical replicates (n=3) and error bars represent 1 SD. Significance was determined by a two-tailed Student's t-test compared to the EzGlc cell-free system: *, p<0.05; ns, p>0.05.

FIGS. 9A-9B. (A) Comparison of protein abundance in tyrosine metabolism (including abbreviated glycolysis, pentose phosphate pathway, shikimate pathway, and aromatic amino acid biosynthesis) between EzGlc and defined medium with aromatic compound dropouts AAA, ACGU and DDGlc. Each bar represents the mean log₂(fold change) in protein abundance in a variant growth medium compared to mean protein abundance in the EzGlc cell-free system, top and bottom of each box represent the largest and smallest fold change observed for a given protein, error bars represent the 90% confidence interval around the mean. Significance was determined by a two-tailed Student's t-test compared to the EzGlc cell-free system: *, p<0.05. Pathway enzymes not depicted can be assumed to have undergone no significant change in abundance. (B) In vitro phenol biosynthesis from ¹³C₆ glucose in a one-pot CFPS-ME reaction measured at 48 hours. Only ¹³C₆ phenol is depicted (m/z=100.1). Values represent averages of technical replicates (n=3) and error bars represent 1 SD. Significance was determined by a two-tailed Student's t-test compared to the EzGlc cell-free system: *, p<0.05; ns, p>0.05.

FIG. 10. Lysates treated with higher bead volumes produce less lactate and more ethanol. Lysates treated with varying volumes of HisPur™ Cobalt beads (Thermo Scientific) were used to prepare CFME reactions with normalized total protein concentrations (4.5 mg/mL). Increasing the ratio of bead volume to lysate volume evidently pulled down more LdhA and PflB protein, resulting in less lactate production and increased flux to ethanol.

FIG. 11. Lysates prepared with optimized source strain cultivation conditions can produce high ethanol yields. Optimization of source strain cultivation conditions (i.e., percentage of glucose in cultivation medium and cell harvesting time) resulted in a lysate with reduced lactate production and improved ethanol yield. When a 1.40 bead/lysate volume ratio is applied, the resulting engineered lysate can synthesize 90 mM EtOH from 46 mM consumed glucose, corresponding to 0.52 g_{EtOH}/g_{Glc} yield.

FIG. 12. Simplified scheme of native (prokaryotes) metabolic pathways suitable for cell-free metabolic engineering (CFME).

FIG. 13. Simplified scheme of native (prokaryotes) and non-native metabolic pathways suitable for cell-free metabolic engineering (CFME). Native pathways are marked in solid boxes, and non-native (heterologous) pathways are marked in dashed boxes.

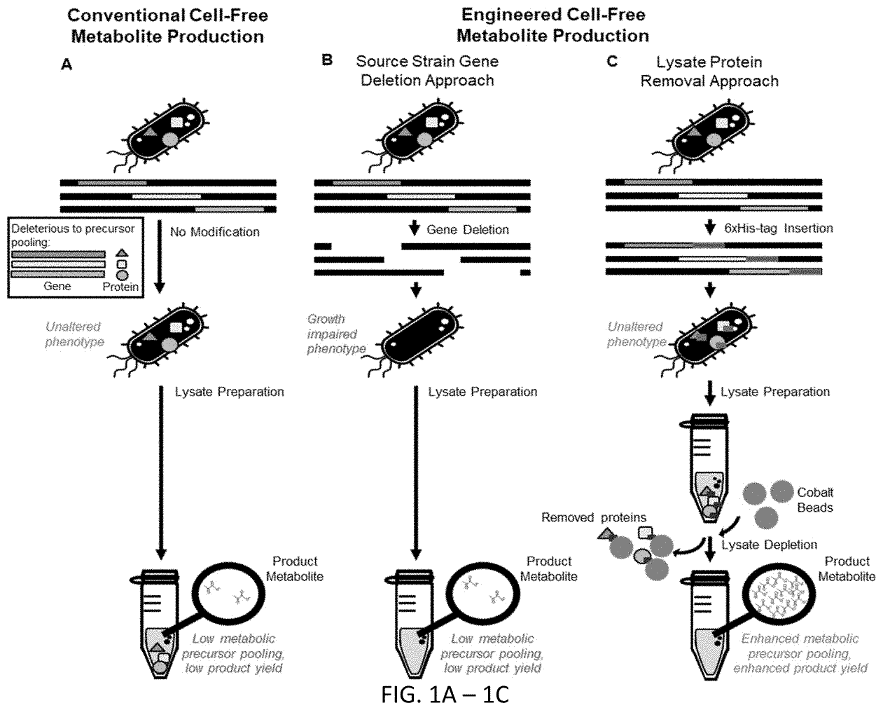

### DETAILED DESCRIPTION

### Definitions

As used herein, the term “about” refers to an approximately +/−10% variation from a given value.

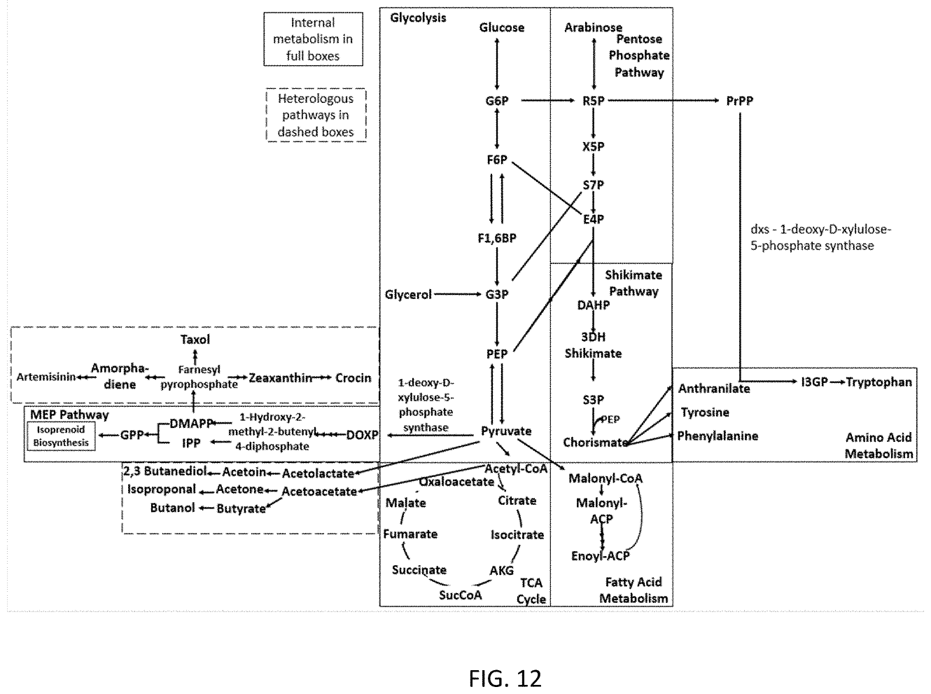

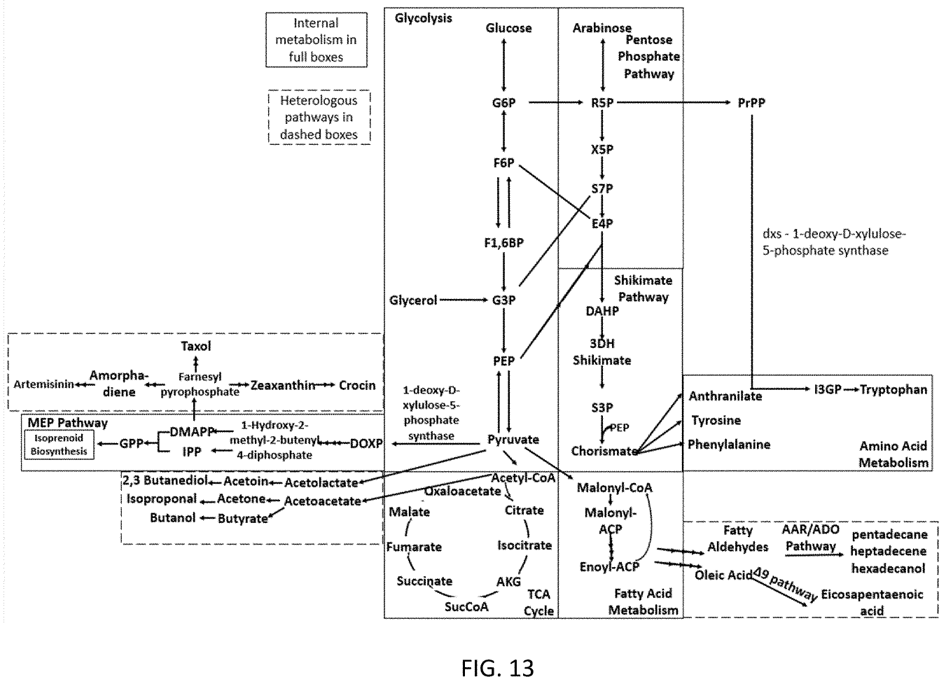

As used herein, the phrase “metabolic flux” refers to the passage of carbon from a carbon source (e.g., amino acids, carbohydrates, nucleic acids, lipids) through a metabolic pathway over time. In some embodiments, metabolic pathways include the glycolysis pathway, the pentose phosphate pathway, the tricarboxylic acid (TCA) cycle, the Shikimate pathway, the 2-C-Methyl-D-erythritol 4-phosphate (MEP) pathway, the amino acid metabolism pathway, and the fatty acid metabolism pathway (including, but not limited to, pathways in FIG. 12 and Kong et al. (Scientific reports, 9.1 (2019): 1-11.)) which is incorporated herein in its entirety). In some embodiments, metabolic pathways include pathways that are non-native (heterologous) to the cell. Exemplary non-native pathways are found in FIG. 13, Yang et al. (Trends in biotechnology (2020), 38(7):745-765) and Roy et al. (Current Opinion in Biotechnology, 50 (2018): 39-46) which are incorporated herein in their entirety.

In a “directed metabolic flux,” the flux of carbon atoms in a cell-free system is channeled towards a metabolite of interest. In some embodiments, the channeling is achieved by removal of enzymes that divert carbon away from the metabolite of interest. For instance, removing 1-deoxy-D-xylulose-5-phosphate synthase to diverts the metabolic flux away from the MEP pathway, and removing pyruvate dehydrogenase (PDH) and/or by removing pyruvate formate-lyase (PDH/PFL) directs the metabolic flux away from Diacetyl-coA production. Either removal, alone or in combination with each other, improves flux towards pyruvate production.

In some embodiments, heterologous enzymes are expressed in the cell (using an exogenous nucleic acid encoding these enzymes) to direct the metabolism to pathways that do not exist in the native cell. In a specific embodiment, heterologous enzymes direct the metabolic flux from pyruvate to the fatty acid metabolism and thereby improves the production of alkanes through heterologous expression of acyl-ACP reductase (AAR) and aldehyde deformylating oxygenase (ADO). See, e.g., FIG. 13., Yang et al. (Trends in biotechnology (2020), 38(7):745-765) and Roy et al. (Current Opinion in Biotechnology, 50 (2018): 39-46) which are incorporated herein in their entirety.)

Pathway and Improve the Production of the Metabolite Pentadecane

As used herein, a “significant impairment” of a metabolism refers to at least 70%, at least 75%, at least 80%, at least 85%, at least 90%, at least 95%, or at least 99% impairment of the cell's metabolism.

As used herein, “substantially” refers to a difference of at least 70%, at least 75%, at least 80%, at least 85%, at least 90%, at least 95%, at least 99% or more as compared to a control.

### Genetically Engineered Cell

As used herein, the term “genetically engineered” (or “genetically modified”) refers to an organism comprising a manipulated genome or nucleic acids.

The present disclosure uses genetically engineered cells to make cell free extracts. In some embodiments, the genetically engineered cell is a prokaryotic cell, a eukaryotic cell or an archeal cell.

In some embodiments, the genetically engineered cell is a prokaryotic cell/organism (a “prokaryote”). In some embodiments, the prokaryote is selected from the genera Lactobacillus, Escherichia, Bacillus, Vibrio, Bifidobacterium, Pseudomonas, Streptomyces and Streptococcus.

In some embodiments, the prokaryote is a strain of Escherichia coli (E. coli). In some embodiments, the E. coli strain is a strain selected from the strains listed in Table 1.

TABLE 1
Exemplary E. coli strains that can be used to prepare
cell free extracts, with genotypes.

| Strain | Genotype |
|---|---|
| BL21-Rosetta | F⁻ ompT hsdS_{B}(r_{B}⁻ m_{B}⁻) gal dcm (DE3)^{a} |
| (DE3)^{B} | pRARE (Novagen) |
| BL21-Rosetta2 | F⁻ ompT hsdS_{B}(r_{B}⁻ m_{B}⁻) gal dcm (DE3)^{a} |
| (DE3)^{B} | pRARE2 (Novagen) |
| BL21-Star | F⁻ ompT hsdS_{B}(r_{B}⁻, m_{B}⁻) gal dcm rne131 |
| (DE3)^{B} | (DE3)^{B} |
| BL21-Gold-dLac | F⁻ ompT hsdS_{B}(r_{B}⁻ m_{B}⁻) dcm gal (DE3)^{a} |
| (DE3)^{B} | endA lacZYA |
| JS006 | MG1655 araC lac1 |
| A19 | rna, gdhA2 relA1 spoT metB1 |
| KC1 | A19 speA tnaA tonA endA sdaA sdaB met⁺ |
| KC6 | KC1 gshA |
| KC6-der. | KC6 rnb ackA⁺ ef-tu⁺ hchA⁺ ibpA⁺ ibpB⁺ |
| | if-1⁺ if-2⁺ if-3⁺ |
| KGK10 | KC6 gorB trxB-HA |
| NMR1 | A19 endA met⁺ |
| NMR2 | A19 speA tnaA tonA endA met⁺ |
| NMR4 | A19 recD endA met⁺ |
| NMR5 | A19 lambda phage < > recBCD met⁺ |
| S30BL/Dna | BL21 (DE3) dnaK/J⁺ grpE⁺ |
| S30BL/DsbC | BL21 (DE3) dsbC⁺ |
| S30BL/GroE | BL21 (DE3) groEL/ES⁺ |
| S30OB | F⁻ ompT hsdSB(r_{B}⁻ m_{B}⁻) gal dcm lacY1 |
| | dhpC (DE3) gor522::Tn10 trxB (Novagen ) |
| S30OB/Dna | S30OB dnaK/J⁺ grpE⁺ |
| S30OB/DsbC | S30OB dsvC⁺ |
| S30OB/GroE | S30OB groEL/ES⁺ |

^{a}Each of these strains is available with or without DE3 modifications, which enables induction of T7 polymerase.

Additional prokaryotes suitable for use in the methods and compositions of the instant disclosure are found in Cole, Stephanie D., et al. (Synthetic and Systems Biotechnology, 5.4 (2020): 252-267), which is incorporated herein in its entirety.

In some embodiments, the genetically engineered cell is a eukaryotic cell. In some embodiments, the eukaryotic cell is selected from a cell from an animal, a cell from a plant, a cell from an insect or a cell from a fungus. Examples of eukaryotic cells suitable for use in this disclosure are found in Hartsough, Emily M., et al. (BioTechniques 59.3 (2015): 149-151), and in Martin, Rey W., et al. (ACS Synthetic Biology, 6.7 (2017): 1370-1379), which are incorporated herein in their entireties.

In some embodiments, the genetically engineered cell is an animal cell selected from a mammalian cell, a fish cell, an amphibian cell, a reptile cell, and a bird cell.

In some embodiments, the mammalian cell is a mammalian cell selected from a human cell, a rabbit cell, a mouse cell, a rat cell, a cat cell and a dog cell. In a specific embodiment, the mammalian cell is from an immortalized cell line, for example a CHO cell or a HeLa cell. In a specific embodiment, the mammalian cell is a rabbit reticulocyte.

In some embodiments, the genetically engineered cell is a plant cell. In a specific embodiment, the plant cell is a plant germ cell. In a specific embodiment, the plant germ cell is a wheat germ cell.

In some embodiments, the genetically engineered cell is a fungus cell selected from the genera Saccharomyces, Pichia, Schizosaccharomyces, Kluyveromyces, and Zygosaccharomyces.

### Affinity Tags and Gene Targeting

As used herein, the phrase “affinity tag” refers to a peptide sequence added to either the N- or C-end of a protein that facilitates purification or removal of the expressed protein. In some embodiments, the affinity tag sequence contains about 5, about 10, about 20, about 30, about 35, about 40, about 45, about 50, about 55, about 60, about 70, about 80, about 90, about 100, about 150, about 200, about 250, or more amino acids.

In some embodiments, an affinity tag is used for removing select proteins from a crude cell lysate post-lysis.

In some embodiments, the affinity tag is selected from a His tag, a FLAG tag, a Strep II tag, a glutathione S-transferase (GST) tag, a Calmodulin binding protein (CBP) tag, a covalent yet dissociable NorpD peptide (CYD) tag, a polyarginine (Poly-Arg or nArg) tag, and a heavy chain of protein C (HPC) tag. Examples of affinity tags that can be used in this disclosure are described in Lichty et al. (Protein Expr. Purif. 41, 98-105), which is incorporated herein in its entirety.

In some embodiments, an affinity tag is added to an enzyme/protein of interest using available gene targeting technologies in the art. Examples of gene targeting technologies include the Multiplex automated genome engineering (MAGE), the Cre/Lox system (described in Kuhn, R., & M. Torres, R., Transgenesis Techniques: Principles and Protocols, (2002), 175-204.), homologous recombination (described in Capecchi, Mario R., Science (1989), 244: 1288-1292), and TALENs (described in Sommer et al., Chromosome Research (2015), 23: 43-55, and Cermak et al., Nucleic Acids Research (2011): gkr218.).

In one embodiment, gene inactivation is achieved by a CRISPR/Cas system. CRISPR-Cas and similar gene targeting systems are well known in the art with reagents and protocols readily available. Exemplary genome editing protocols are described in Jennifer Doudna, and Prashant Mali, “CRISPR-Cas: A Laboratory Manual” (2016) (CSHL Press, ISBN: 978-1-621821-30-4) and Ran, F. Ann, et al. Nature Protocols (2013), 8 (11): 2281-2308; and Li, Y., Lin, Z., Huang, C., Zhang, Y., Wang, Z., Tang, Y. jie, Chen, T., and Zhao, X. (2015) “Metabolic engineering of Escherichia coli using CRISPR-Cas9 meditated genome editing”. Metab. Eng. 31, 13-21, which are incorporated herein in their entireties.

### Controlled Growth Media Conditions

As used herein, the phrase “controlled growth medium” refers to a solid, liquid or semi-solid designed to support the growth of a cell or a population of cells via the process of cell proliferation in which a parameter, such as medium ingredients, pH, temperature or oxygenation, has been specifically altered.

Numerous metabolites are used by cells to assist their growth, activity, and function. The availability of these metabolites in the growth medium influences a cell's requirement to devote resources to produce these same or related precursor materials. Therefore, the presence or absence of these metabolites from the growth medium can cause cells to decrease or increase the activity of the pathways, and associated enzymes, necessary to produce specific metabolites. In general, the present inventors have developed controlled growth media, by including or removing selected metabolites from growth media. In cells grown in controlled growth media, cellular energy and resources will be shifted either towards or away from the production pathway for the missing or included metabolite and thus flux towards the metabolite will be changed in the derived cell extract.

In some embodiments, the controlled growth medium does not have aromatic amino acids (i.e., amino acids phenylalanine, tryptophan and tyrosine). Cells grown in controlled growth media lacking aromatic amino acids display improved production of aromatic compounds (such as phenylpropanoids) in the resulting cell-free system.

In some embodiments, the controlled growth medium does not have branched amino acids (i.e., amino acids valine, leucine and isoleucine). Cells grown in controlled growth media lacking branched amino acids display improved production of branch-chained molecules (e.g., branch-chained alcohols) and fatty acids in the resulting cell-free system.

In some embodiments, the controlled growth medium comprises and organic hydrocarbon. In some embodiments, the organic hydrocarbon is selected from phenol, toluene, pinene, benzene, ethylbenzene, naphtalene or limonene. Additional examples of organic hydrocarbons are also found in Sikkema, Jan et al. (Microbiological Reviews, 59.2 (1995): 201-222), which is incorporated herein in its entirety. To cope with membrane stress, cells grown in a controlled growth medium containing an organic hydrocarbon are enriched in enzymes that catalyze fatty acid trans-isomerization, thus facilitating the derivatization of fatty acids in the resulting cell-free system.

The inventors have also recognized that cellular metabolism and the metabolic proficiencies of the derived cell extract are, likewise, altered by changes in the cellular environment. Cellular metabolism shifts with changes in temperature, pH, oxygenation and growth state, among others. Metabolic pathway activity and the abundance of associated enzymes can be tuned by manipulating the environmental conditions of cell growth.

In some embodiments, the controlled growth medium has a predefined temperature.

In some embodiments, the controlled growth medium has a low temperature. As used herein, the phrase “low temperature” refers to a temperature less than 30° C. In some embodiments, the controlled growth medium has a temperature of about 28° C., about 27° C., about 25° C., about 20° C., about 15° C., about 10° C., about 5° C., or about 3° C.

In some embodiments, the controlled growth medium has a high temperature. As used herein, the phrase the phrase “high temperature” refers to a temperature more than 30° C. In some embodiments, the controlled growth medium has a temperature of about 32° C., about 35° C., about 38° C., about 40° C., or about 45° C.

In some embodiments, the controlled growth medium has a predefined pH or a predefined pH range.

In some embodiments the controlled growth medium has an acidic (low) pH. As used herein, the phrase “acidic pH” refers to a pH less than 7. In some embodiments, the controlled growth medium comprises a pH of about 6, a pH of about 5, a pH of about 4, or a pH of about 2 or lower. In some embodiments, cells grown in a growth medium having low pH are enriched with the enzyme glutamate decarboxylase facilitating synthesis of the neurotransmitter GABA (gamma-aminobutyric acid) in the resulting cell-free system.

In some embodiments the controlled growth medium has an alkaline (high) pH. As used herein, the phrase “alkaline pH” refers to a pH more than 7. In some embodiments, the controlled growth medium comprises a pH of about 7.5, a pH of about 8, a pH of about 9, a pH of about 10, a pH of about 11, a pH of about 12, a pH of about 13, or a pH of about 14 or higher.

In some embodiments, the controlled growth medium is a liquid medium that has a predefined oxygenation level.

In some embodiments, the controlled growth medium has a low oxygen level.

As used herein, the phrase “low oxygen level” refers to less than about 8% dissolved oxygen by mass. In some embodiments, the controlled growth medium comprises about 8%, about 7.5%, about 7%, about 6.5%, about 6%, about 5.5%, about 5%, about 4.5%, about 4%, about 3.5%, about 3%, about 2.5%, about 2% dissolved oxygen or less.

In some embodiments, the controlled growth medium has an oxygen level higher than 8%. In some embodiments, the controlled growth medium comprises about 8.5%, about 9%, about 9.5%, about 10%, about 10.5%, about 11%, about 12.5%, about 13%, about 13.5%, about 14%, about 14.5%, about 15%, about 15.5%, about 16%, about 16.5%, about 17% dissolved oxygen, or more.

An Enzyme that Affects the Amount of a Metabolite of Interest

In some embodiments, the metabolite of interest is a metabolite in a cellular pathway. In some embodiments, the metabolite of interest is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, and a metabolite in the fatty acid metabolism pathway. In some embodiments, the metabolite of interest is a native metabolite that can be produced by a wild-type or genetically non-modified cell. In some embodiments, the

In some embodiments, the metabolite of interest is selected from pyruvate, ethanol, mevalonate, isopentyl pyrophosphate and acetyl coenzyme A.

As used herein, the phrase an “enzyme that affects the amount of a metabolite of interest” refers to an enzyme that affects construction or destruction of the metabolite of interest. In some embodiments, the “enzyme that affects the amount of a metabolite of interest” is connected to a pathway that affects construction or destruction of the metabolite of interest.

In some embodiments, the “enzyme that affects the amount of a metabolite of interest” uses the metabolite of interest as a substrate, and converts it to another molecule, thereby reducing the amount of the metabolite of interest.

In some embodiments, the “enzyme that affects the amount of a metabolite of interest” uses a precursor of the metabolite of interest as a substrate, thereby competing with the production of the metabolite of interest by diverting the metabolic flux away from the productions of the metabolite of interest and reducing the amount of the metabolite of interest.

In some embodiments, the “enzyme that affects the amount of a metabolite of interest” increases the amount of precursor of the metabolite of interest, thereby increasing the amount of the metabolite of interest.

In some embodiments, the “enzyme that affects the amount of a metabolite of interest” affects the amount of the metabolite by changing the pH of the cell and resulting cell-free extract.

In some embodiments, the “enzyme that affects the amount of a metabolite of interest” is 1, 2, 3, or 4 reactions upstream of the metabolite of interest in the metabolic pathway that produces the metabolite of interest. In some embodiment, the “enzyme that affects the amount of a metabolite of interest” is immediately downstream of the metabolite of interest in the metabolic pathway that produces the metabolite of interest.

In some embodiments, the “enzyme that affects the amount of a metabolite of interest” is selected from an enzyme in the glycolysis pathway, an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, and an enzyme in the fatty acid metabolism pathway.

### Methods for Directing Metabolic Flux in a Cell

Inventors of the instant disclosure have found that it is possible to direct metabolic flux of a cell towards a specific metabolite of interest by removing certain enzymes from the cell, or its cell-free lysate. The inventors achieved this by adding affinity tags to the enzymes to be removed from the cell or cell lysate.

An aspect of this disclosure is directed to a method comprising linking an affinity tag to at least one enzyme in the cell that affects the amount of a metabolite of interest.

In some embodiments, the method comprises linking the affinity tag to multiple or all enzymes that affect the amount of the metabolite.

In some embodiments, the at least one enzyme is a central metabolism enzyme (aka. an “essential enzyme”). As used herein, a “central metabolism enzyme” is an enzyme that its deletion or inactivation significantly impairs the cell's metabolism or kills the cell. In some embodiments, deletion or inactivation of the at least one enzyme significantly impairs the cell's metabolism or kills the cell. A non-limiting list of essential genes in prokaryotes are found in Kong et al. (Scientific reports, 9.1 (2019): 1-11.)), incorporated herein in its entirety.

In some embodiments, the method further comprises expressing in the cell a nucleic acid encoding an exogenous enzyme that affects the concentration of the metabolite.

In some embodiments, the exogenous enzyme is an enzyme that is not native to the cell (i.e., the exogenous enzyme is from a different species). In some embodiments, the non-native exogenous enzyme adds the cell a non-native metabolic pathway that results in a change in the concentration of the metabolite of interest.

In some embodiments, the exogenous enzyme increases the amount of precursor of the metabolite of interest, thereby increasing the amount of the metabolite of interest.

In some embodiments, the exogenous enzyme is an engineered version of a native enzyme. In some embodiments, the engineered version of the enzyme is constitutively active. In some embodiments, the engineered version of the enzyme is catalytically dead, dominant negative version of the native enzyme.

In some embodiments, the nucleic acid encoding an exogenous enzyme is codon optimized.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase and prenyl transferase.

In some embodiments, the metabolite is acetyl coenzyme A, and wherein the enzyme is pyruvate dehydrogenase.

In some embodiments, the genetically engineered cell is a bacterium from genus Escherichia, the metabolite is pyruvate and the at least one enzyme is selected from PpsA, PflB, AceE and LdhA. In some embodiments, wherein each of PpsA, PflB, AceE and LdhA is linked to the affinity tag.

### Methods for Making Reduced Cell-Free Extracts

Another aspect of the disclosure is directed to a method for making a cell-free extract that has a directed metabolic flux towards a metabolite of interest comprising: growing a genetically engineered cell under conditions that allow production of the metabolite, wherein at least one enzyme in the genetically engineered cell that affects the amount of metabolite has been engineered to be linked to an affinity tag; making a crude cell extract from the genetically engineered cell; removing the at least one enzyme from the crude cell extract using affinity purification, thereby obtaining a cell-free extract capable of producing the metabolite.

In some embodiments, multiple or all enzymes that affect the amount of the metabolite have been engineered to be linked to an affinity tag and have been substantially removed from the cell extract.

In some embodiment, the at least one enzyme is a central metabolism enzyme that, deletion or inactivation of the at least one enzyme significantly impairs the cell's metabolism or kills the cell.

In some embodiments, the genetically engineered cell further comprises a nucleic acid encoding an exogenous enzyme that affects the concentration of the metabolite.

In some embodiments, the at least one enzyme is selected from an enzyme in the glycolysis pathway, an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, and an enzyme in the fatty acid metabolism pathway.

In some embodiments, the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, and a metabolite in the fatty acid metabolism pathway.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase and prenyl transferase.

In some embodiments, the metabolite is acetyl coenzyme A, and wherein the enzyme is pyruvate dehydrogenase.

In some embodiments, the genetically engineered cell is a bacterium from genus Escherichia, the metabolite is pyruvate and the at least one enzyme is selected from PpsA, PflB, AceE and LdhA. In some embodiments, each of PpsA, pflB, AceE and LdhA is linked to the affinity tag.

Cell-Free Extracts with Directed Metabolism

Another aspect of the disclosure is directed to cell free extracts that have directed metabolic flux towards a metabolite of interest. In cell free extracts that have directed metabolic flux, pathways that lead to less production of the metabolite of interest (e.g., by competing with the production of the metabolite of interest, or by directly using up the metabolite of interest) are substantially removed from the cell extract.

In some embodiments, the cell-free extract comprises an extract from a genetically engineered cell, wherein at least one enzyme that affects the amount of the metabolite has been substantially removed from the cell extract. In some embodiments, multiple or all enzymes that affect the amount of the specific metabolite have been substantially removed from the cell extract.

In some embodiments, the at least one enzyme is a central metabolism enzyme that, deletion or inactivation of the at least one enzyme significantly impairs the cell's metabolism or kills the cell.

In some embodiments, the genetically engineered cell further comprises a nucleic acid encoding an exogenous enzyme that affects the concentration of the metabolite.

In some embodiments, the at least one enzyme is selected from an enzyme in the glycolysis pathway, an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, and an enzyme in the fatty acid metabolism pathway.

In some embodiments, the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, and a metabolite in the fatty acid metabolism pathway.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase and prenyl transferase.

In some embodiments, the metabolite is acetyl coenzyme A, and wherein the enzyme is pyruvate dehydrogenase.

In some embodiments, the genetically engineered cell is a bacterium from genus Escherichia, the metabolite is pyruvate and the at least one enzyme is selected from PpsA, PflB, AceE and LdhA. In some embodiments, each of PpsA, PflB, AceE and LdhA is linked to the affinity tag.

In some embodiments, the genetically engineered cell has been cultured in a controlled growth medium before extract preparation. In some embodiments, the controlled growth medium lacks aromatic amino acids or comprises an organic hydrocarbon. In some embodiments, the controlled growth medium comprises a pre-defined temperature, pH, or oxygenation level.

### Kits

Another aspect of the disclosure is directed to a kit comprising: a cell-free extract that has a directed metabolic flux towards a metabolite of interest comprising a reduced extract from a genetically engineered cell, wherein at least one enzyme that affects the amount of the metabolite has been substantially removed from the cell extract.

In some embodiments, multiple or all enzymes that affect the amount of the specific metabolite have been substantially removed from the cell extract.

In some embodiments, the at least one enzyme is a central metabolism enzyme that, deletion or inactivation of the at least one enzyme significantly impairs the cell's metabolism or kills the cell.

In some embodiments, the genetically engineered cell further comprises a nucleic acid encoding an exogenous enzyme that affects the concentration of the metabolite.

In some embodiments, the at least one enzyme is selected from an enzyme in the glycolysis pathway, an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, and an enzyme in the fatty acid metabolism pathway.

In some embodiments, the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, and a metabolite in the fatty acid metabolism pathway.

In some embodiments, the metabolite is isopentyl pyrophosphate, and wherein the enzyme is selected from geranyl pyrophosphate synthase, farnesyl pyrophosphate synthase, geranylgeranyl pyrophosphate synthase and prenyl transferase.

In some embodiments, the metabolite is acetyl coenzyme A, and wherein the enzyme is pyruvate dehydrogenase.

In some embodiments, the genetically engineered cell is a bacterium from genus Escherichia, the metabolite is pyruvate and the at least one enzyme is selected from PpsA, PflB, AceE and LdhA. In some embodiments, each of PpsA, PflB, AceE and LdhA is linked to the affinity tag.

In some embodiments, the genetically engineered cell has been cultured in a controlled growth medium before extract preparation. In some embodiments, the controlled growth medium lacks aromatic amino acids or comprises an organic hydrocarbon. In some embodiments, the controlled growth medium comprises a pre-defined temperature, pH, or oxygenation level.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

### Generation and Validation of Genome Engineered Strains Using MAGE

All multiplex allele-specific PCR (MASC-PCR), Sanger Sequencing oligos, and recombineering oligos were created manually and ordered from IDT (Coralville, Iowa) with standard purification. Each targeting oligo incorporated four phosphorothioated bases on the 5′ terminus. An 18-base CACCATCACCATCACCAT sequence was used to add the 6×His-tag and directed at either the N- or C-terminus based on previous literature or crystal structure analysis. The pORTMAGE protocol used in this study followed previous work with the exception that growth was carried out in 6 mL of Luria-Bertani-Lennox (lbl) cultures in glass tubes with 100 mg/mL of carbenicillin, recovery was performed in 3 mL of terrific broth with a 1-hour incubation time prior to adding 3 mL of lbl-carb for outgrowth. Given the significant time required to find accumulated mutations in a single strain, the additive mutations were started from previously found mutations such that Δ1 was used to create Δ2 and so on as per the protocols used in previous studies. After every 8-12 cycles of MAGE, 30-60 colonies were screened for genome edits using MASC-PCR as detailed previously. Allelic genotyping was performed using standard primers designed to flank both modified genes. Amplicons were Sanger sequenced to validate the insertion of the 6×His-tag sequence. Primer sequences used in this study are listed in Table 1 and Table 2.

### Cell-Free Extract Preparation Protocol

Following plasmid curing, the cell extracts were prepared from E. coli BL21 Star (DE3) grown at 37° C. in 2×YPT-G (16 g L-1 tryptone, 10 g L-1 yeast extract, 5 g L-1 NaCl, 7 g L-1 KH2PO4, 3 g L-1 K₂HPO₄, 18 g L-1 glucose). Cell extracts were prepared by harvesting 50-mL cultures grown in baffled Erlenmeyer flasks to an OD₆₀₀ of 5.0. Cells were harvested by centrifugation at 5000×g for 10 min in 50 mL volumes and washed twice with S30 buffer (14 mM magnesium acetate, 60 mM potassium acetate, 1 mM dithiothreitol (DTT) and 10 mM Tris-acetate, pH 8.2) by resuspension and centrifugation. The pellets were weighed, flash-frozen, and stored at −80° C. Extracts were prepared by thawing and resuspending the cells in 0.8 mL of S30 buffer per gram of cell wet weight. The resuspension was lysed using 530 joules per mL of suspension at 50% tip amplitude with ice water cooling. Following sonication, tubes of cell extract were centrifuged twice at 21,100×g for 10 minutes at 4° C., aliquoted, frozen with liquid nitrogen, and stored at −80° C.

### Cell-Free Extract Depletions

Cell extracts were depleted for specific proteins by adding one volume of cell extract to 0.2× volume of ice-cold HisPur™ Cobalt Resin (ThermoFisher Scientific) suspension in 1.5 mL microcentrifuge tubes. Prior to the addition of lysate, HisPur™ Cobalt Resin was washed 2× with 500 μL S30 buffer and incubated with 10 mM imidazole buffer (pH 4.5; 10 mM imidazole, 50 mM monopotassium chloride, 300 mM NaCl). Lysate-resin mixtures were incubated for 1 hour at 4° C. under shaking conditions (800 rpm) to ensure the suspension of the resin particles in the extracts and then centrifuged at 14,000×g for 30 seconds. Supernatants were aliquoted, flash-frozen, and stored at −80° C. until used. His-tagged proteins were eluted from the HisPur™ Cobalt Resin by suspending the resin in 50 μL elution buffer (pH 4.5; 250 mM imidazole, 50 mM monosodium phosphate, 300 mM NaCl) for 30 minutes at 4° C. under shaking conditions (800 rpm). The eluate was obtained for proteomic quantification by spinning down the suspension at 14,000×g for 30 seconds and collecting the supernatant. The selective depletions were verified with an anti-6×His Western Blot.

### CFME Reaction Set-up

Glucose consumption reactions were carried out at 37° C. in 50 μL volumes using a solution of 100 mM glucose, 18 mM magnesium glutamate, 15 mM ammonium glutamate, 0.2 mM Coenzyme A, 195 mM potassium glutamate, 1 mM ATP, 150 mM Bis-Tris, 1 mM NAD+, 10 mM dipotassium phosphate. Similarly, pyruvate fed reactions were carried out using the same conditions with the exception of 25 mM pyruvate being used in place of glucose. Extracts were added to a final protein concentration of 4.5 mg mL-1. Each reaction was quenched by the addition of 50 μL of 5% TCA. The supernatant was centrifuged at 11,000×g for 5 minutes and directly used for analytical measurements.

### Proteomics Sample Preparation

Samples of both depleted and nondepleted versions of WT, 6×His-pflB, 6×His-2, 6×His-3, and 6×His-4 cell extracts were each prepared in triplicate as follows. Extracts were solubilized in 200 μL of 4% SDS in 100 mM Tris buffer, pH 8.0. Trichloroacetic acid was added to achieve a concentration of 20% (w/v). Samples were vortexed and incubated at 4° C. for 2 h followed by 10 min at −80° C. Samples were then thawed on ice prior to centrifugation (˜21,000 g) for 10 min at 4° C. to pellet precipitated proteins from the detergent and solutes. The supernatant was discarded, and samples were washed with 1 mL of ice-cold acetone. Pelleted proteins were then air-dried and resuspended in 100 μL of 8 M urea in 100 mM Tris buffer, pH 8.0. Proteins were reduced with 10 mM dithiothreitol incubated for 30 min and alkylated with 30 mM iodoacetamide for 15 min in the dark at room temperature. Proteins were digested with two separate and sequential aliquots of sequencing grade trypsin (Promega) of 1 μg. Samples were diluted to 4 M urea and digested for 3 hours, followed by dilution to 2 M urea for overnight digestion. Samples were then adjusted to 0.1% trifluoroacetic acid and then desalted on Pierce peptide desalting spin columns (Thermo Scientific) as per manufacturer's instructions. Samples were vacuum-dried with a SpeedVac (Thermo Scientific) and then resuspended in 50 μL of 0.1% formic acid. Peptide concentrations were then measured using a NanoDrop spectrophotometer (Thermo Scientific) and 2 μg of each sample was used for LC-MS measurement.

### LC-MS/MS Analysis

All samples were analyzed on a Q Exactive Plus mass spectrometer (Thermo Scientific) coupled with an automated Vanquish UHPLC system (Thermo Scientific). Peptides were separated on a triphasic precolumn (RP-SCX-RP; 100 μm inner diameter and 15 cm total length) coupled to an in-house-pulled nanospray emitter of 75 μm inner diameter packed with 25 cm of 1.7 μm of Kinetex C18 resin (Phenomenex). For each sample, a single 2 μg injection of peptides were loaded and analyzed across a salt cut of ammonium acetate (500 mM) followed by a 210 min split-flow (300 nL/min) organic gradient, wash, and re-equilibration: 0% to 2% solvent B over 27 min, 2% to 25% solvent B over 148 min, 25% to 50% solvent B over 10 min, 50% to 0% solvent B over 10 min, hold at 0% solvent B for 15 min. MS data were acquired with the Thermo Xcalibur software using the top 10 data-dependent acquisition.

### Proteome Database Search

All MS/MS spectra collected were processed in Proteome Discoverer, version 2.3 with MS Amanda and Percolator. Spectral data were searched against the most recent E. coli reference proteome database from UniProt to which mutated sequences and common laboratory contaminants were appended. The following parameters were set up in MS Amanda to derive fully tryptic peptides: MS1 tolerance=5 ppm; MS2 tolerance=0.02 Da; missed cleavages=2; Carbamidomethyl (C, +57.021 Da) as static modification; and oxidation (M, +15.995 Da) as dynamic modifications. The Percolator false discovery rate threshold was set to 1% at the peptide-spectrum match and peptide levels. FDR-controlled peptides were then quantified according to the chromatographic area-under-the-curve and mapped to their respective proteins. Areas were summed to estimate protein-level abundance.

### Tags and Genomic Engineering

The inventors of the instant disclosure opted to utilize the 6×His tag because of its inexpensive compatible resins, which make 6×His tag affinity purification a widely accessible method. However, any other suitable tag (e.g., FLAG and HPC) can be used for pull-downs from the lysate proteome albeit at higher costs. Among several tags that have been extensively reviewed across different model organisms including E. coli, Strep II tags are considerably highly selective for a moderate expense.

The small size (18 bp) of the 6×His tag relative to other tags (˜24-1200 bp) also made it an excellent choice for MAGE enabled genome engineering, which is naturally limited to small sequence edits such as SNPs. However, the claimed lysate engineering method is not limited to the use of MAGE as a tool for the genomic insertion of affinity tags. Other multiplex genome engineering methods that efficiently allow large genomic insertions have recently advanced. While the inventors show that MAGE reasonably allows for the insertion of the 18 bp 6×His-tag into four sites of the genome over multiple iterations, this method combined with CRISPR technology has enabled the insertion of even larger sequences into bacterial genomes with high editing efficiency. Li et al. (2015, Metab. Eng. 31, 13-21) reported the incorporation of a single 2 kb dsDNA fragment in >90% of an E. coli population in one cycle. An emerging model system for biotechnology, Vibrio natriegens, is naturally amenable to large genomic insertions in a multiplex fashion, which allows for the insertion of 3-4 6 kb gene fragments in 25% of the population over a single iteration (described in Daila, T N. et al., ACS Synth. Biol. 6, 1650-1655, which is incorporated herein in its entirety). The inventors, therefore, expect that combinations of more efficient genome engineering tools and larger affinity tags could enhance the approach described herein and enable the rapid manipulation of lysate metabolism.

### Proteomic Data Analysis

For differential abundance analysis of proteins, the protein table was exported from Proteome Discoverer. Proteins were filtered to remove stochastic sampling. All proteins present in 2 out of 3 biological replicates in any condition were considered valid for quantitative analysis. Data was log₂ transformed, LOESS normalized between the biological replicates and mean-centered across all the conditions. Missing data were imputed by random numbers drawn from a normal distribution (width=0.3 and downshift=2.8 using Perseus software (the Perseus website). The resulting matrix was subjected to ANOVA and a post-hoc TukeyHSD test to assess protein abundance differences between the different experimental groups. The statistical analyses were done using an in-house developed R script.

### Metabolite Measurements

Glucose, pyruvate, lactate, acetate, formate, and ethanol measurements were performed via high-performance liquid chromatography (HPLC) with an Agilent 1260 equipped with an Aminex HPX 87-H column (Bio-Rad, Hercules, Calif.). Analytes were eluted with isocratic 5 mM sulfuric acid at a flow rate of 0.55 mL min-1 at 35° C. for 25 mM Metabolite concentrations were calculated from measurements collected through a refractive index detector (Agilent, Santa Clara, Calif.) and a diode array UV-visible detector (Agilent, Santa Clara, Calif.) reading at 191 nm. Pyruvate, glucose, lactate, acetate, formate, and ethanol standards were used for sample quantification using linear interpolation of external standard curves.

### Oligos

TABLE 2
MAGE oligos use for this study (first four
bases in each oligo are phosphorothioated)

| Primer | Sequence |
|---|---|
| Pfl | aataaaaaatccacttaagaaggtaggtgttacatgCAC |
| | catCACcatCACCATtccgagcttaatgaaaagttagcc |
| | acagcctgggaa (SEQ ID NO: 1) |
| Ldh | taaatgtgattcaacatcactggagaaagtcttatgCAC |
| | catCACcatCACCATaaactcgccgtttatagcacaaaa |
| | cagtacgacaag (SEQ ID NO: 2) |
| Ppsa | caaaccgttcatttatcacaaaaggattgttcgatgCAC |
| | catCACcatCACCATtccaacaatggctcgtcaccgctg |
| | gtgctttggtat (SEQ ID NO: 3) |
| Pdh | actcaacgttattagatagataaggaataacccatgCAC |
| | catCACcatCACCATtcagaacgtttcccaaatgacgtg |
| | gatccgatcgaa (SEQ ID NO: 4) |

TABLE 3
MASC-PCR oligos used for this study.

| Primer | Sequence |
|---|---|
| Pfl F | GCCAGCCAGGAAGGACTCGTCACCCTCG (SEQ ID NO: 5) |
| Pfl R | GCAGTAAATAAAAAATCCACTTAAGAAGGTAGGTGTTACATGC |
| | (SEQ ID NO: 6) |
| Ldh F | CAGCGTCATCATCATACCGATGGC (SEQ ID NO: 7) |
| Ldh R | CTTAAATGTGATTCAACATCACTGGAGAAAGTCTTATGC |
| | (SEQ ID NO: 8) |
| Ppsa F | GCTGGTTTACGCCGCTTTGGTCC (SEQ ID NO: 9) |
| Ppsa R | ACCGTTCATTTATCACAAAAGGATTGTTCGATGC |
| | (SEQ ID NO: 10) |
| Pdh F | TGGCCTTTATCGAAGAAATTTTGCTCGACAG |
| | (SEQ ID NO: 11) |
| Pdh R | ATCCACGTCATTTGGGAAACGTTCTGAA |
| | (SEQ ID NO: 12) |

### Cell Extract Preparation

Cell extracts were prepared from E. coli BL21 Star (DE3) grown at 37° C. in variants of YTPG (16 g L-1 tryptone, 10 g L⁻¹ yeast extract, 5 g L⁻¹ NaCl, 7 g L⁻¹ KH₂PO₄, 3 g L-1 K₂HPO₄, 18 g L⁻¹ glucose) and EZ Rich medium. EZ Rich medium was made from amino acid EZ supplement (0.8 mM L-Alanine, 5.2 mM L-Arginine HCl, 0.4 mM L-Asparagine, 0.4 mM L-Aspartic Acid, 0.6 mM L-Glutamic Acid, 0.6 mM L-Glutamine, 0.8 mM L-Glycine, 0.2 mM L-Histidine, 0.4 mM L-Isoleucine, 0.4 mM L-Proline, 10 mM L-Serine, 0.4 mM L-Threonine, 0.1 mM L-Tryptophan, 0.6 mM L-Valine, 0.8 mM L-Leucine, 0.4 mM L-Lysine, 0.2 mM L-Methionine, 0.4 mM L-Phenylalanine, 0.1 mM L-Cysteine, 0.2 mM L-Tyrosine, 0.01 mM Thiamine HCl, 0.01 mM calcium pantothenate, 0.01 mM para-amino benzoic acid, 0.01 mM para-hydroxy benzoic acid, 0.1 mM 2,3-dihydroxy benzoic acid), nucleotide (199 μM adenine, 199 μm cytosine, 199 μM uracil, 199 μM guanine, 1.5 mM potassium hydroxide) and buffer solutions (4 mM tricine, 10 μM iron sulfate, 9.5 mM ammonium chloride, 276 μM potassium sulfate, 0.5 μM calcium chloride, 525 μM magnesium chloride, 50 mM sodium chloride, 2.92×10⁻⁷ mM ammonium molybdate, 4.00×10⁻⁵ mM boric acid, 3.02×10⁻⁶ mM cobalt chloride, 9.62×10⁻⁷ mM cupric sulfate, 8.08×10⁻⁶ mM manganese chloride, 9.74×10⁻⁷ mM zinc sulfate, 1.32 mM potassium phosphate dibasic). EZ Rich defined rich medium kit was supplied by Teknova and purchased from VWR (Radnor, Pa., USA). 5× EZ Supplement without tyrosine, tryptophan and phenylalanine was purchased from BioWorld (Atlanta, Ga., USA). Variants of EZ Rich medium and their designations are summarized in Table 5. In brief, for preparation of cell extracts, 50 mL cultures were grown in baffled 250 mL Erlenmeyer flasks to an OD600 of −0.8 and induced to 1 mM isopropyl-β-d-thiogalactopyranoside. Cells were harvested 2.5 hours after induction, corresponding to OD600 of 2.8, 3.6 and 4.0 for media YTPG, EZ Rich, and EzGlc, respectively. EzGlc variants were harvested at a defined time (2.5 hours) after induction. Cells were harvested by centrifugation at 5000×g for 10 min and washed with S30 buffer (2×, 25 mL, 14 mM magnesium acetate, 60 mM potassium glutamate, 1 mM dithiothreitol and 10 mM Tris-acetate, pH 8.2). Cell pellets were weighed, flash-frozen in liquid nitrogen, and stored at −80° C. For extract preparation, cells were thawed and resuspended in 0.8 mL of S30 buffer per mg of cell wet weight before lysis with a Branson Ultrasonics Sonifier SFX250 equipped with a microprobe. Cells were lysed with 530 joules per mL of suspension at 50% tip amplitude in a 0° C. water bath. Post-lysis the cell-slurry was centrifuged twice for 10 minutes at 21,100×g at 4° C., the supernatant was aliquoted, flash-frozen and stored at −80° C.

### Cell-Free Reactions

Cell-free reactions for protein synthesis or phenol production were carried out at 30° C. in 25 μL volumes with the following components: 40 mM ¹³C₆ glucose, 1.2 mM ATP; 0.85 mM each of GTP, UTP and CTP; 34 μg/mL folinic acid; 67.7 mM creatine phosphate, 3 μg/mL creatine kinase, 0.4 mM pyridoxal 5′-phosphate, 2 mM each of the 20 translatable amino acids, 0.33 mM nicotinamide adenine dinucleotide (NAD), 0.26 mM coenzyme A (CoA), 33 mM PEP, 18 mM magnesium glutamate, 15 mM ammonium glutamate, 195 mM potassium glutamate, 1.5 mM spermidine, 1 mM putrescine, 57 mM Bis-Tris pH 7, 10 ng/μL plasmid DNA and 15 μL cell extract adjusted to 10 mg/mL by Bradford assay. Cell-free reactions were overlaid with 100 μL of tributyrin to prevent evaporation. Cell-free protein synthesis of sfGFP was performed in a 96 well plate in a Perkin Elmer EnSpire 2300 for 8 hours, with fluorescent measurements (excitation 488 nm, emission 509 nm) every 20 minutes. Phenol production reactions were run for 48 hours in 1.5 mL microcentrifuge tubes. After 48 hours, phenol production reactions were vortexed and centrifuged for 10 minutes at 21,100×g at 4° C. 50 μL of tributyrin overlay was removed, added to 0.5 mL of dicholoromethane and subjected to analysis by GCMS.

### Phenol Quantitation

In vitro synthesized phenol was quantified on an Agilent 7890A gas chromatograph equipped with a 5975C mass spectrometer. Tributyrin overlays diluted with dichloromethane were injected onto a HP-5MS column at 40° C. Initial oven temperature was held for 3 minutes, ramped to 120° C. at 22° C./min and held for 1 additional minute. The oven was then heated to 325° C. and maintained for 3 minutes. ¹³C₆, ¹³C₄, and non-labeled phenol were monitored at m/z 100.1, 98.1, and 94.1 respectively. Phenol was quantified by peak integration and comparison to a standard curve in Thermo Xcalibur. Three technical replicates and two injection replicates were measured for every sample.

### Statistical Analysis

At least three biological replicates were used for all proteomics measurements. Differences in protein abundance, based upon average log₂ protein intensity, were determined by Student's T test (2-tailed, unpaired, equal variance). P-values for hypothesis generation were calculated without adjustment51. Two p-value thresholds were used in this work and depended on the number of proteins being compared. A more stringent threshold (p<0.01) was used for comparisons between the >1200 proteins found in the lysate along with a fold-change cut off. The more rigorous cut-off is necessary due to the large number of comparisons. A less stringent threshold (p<0.05) was used when comparing proteins that comprised the phenol biosynthesis pathway, without a fold-change cut-off, to assess for even small changes in this subset of proteins. Statistics were performed, and plots were generated in R (version 3.5.3) with packages Tidyverse and ggpubr52.

### Abbreviations

G6P, glucose 6-phosphate; F6P, fructose 6-phosphate; F1,6BP, fructose 1,6-bisphosphate; G3P, glyceraldehyde-3-phosphate; PEP, phosphoenolpyruvate; RSP, ribose 5-phosphate; XSP, xylulose 5-phosphate; S7P, sedoheptulose 7-phosphate; E4P, erythrose 4-phosphate; PrPP, phosphoribosyl pyrophosphate; DAHP, 3-deoxy-D-arabinoheptulosonate 7-phosphate; 3DHS, 3-dehydroshikimate; S3P, shikimate 3-phosphate; I3GP, indole-3-glycerol phosphate. Enzyme abbreviations with Enzyme Commission numbers: G6PDH, glucose 6-phosphate dehydrogenase (EC 1.1.1.49); AraA, arabinose isomerase (EC 5.3.1.4); PRPPS, phosphoribosyl pyrophosphate synthase (EC 2.7.6.1); Rpe, ribulose 5-phosphate 3-epimerase (EC 5.1.3.1); TktA, transketolase 1 (EC 2.2.1.1); FBPase I, fructose 1,6-bisphosphotase class I (EC 3.1.3.11); FBPase II, fructose 1,6-bisphosphotase class II (EC 3.1.3.11); GlyK, glycerol kinase (EC 2.7.1.30); GapC, glyceraldehyde-3-phosphoate dehydrogenase (EC 1.2.1.12); PEPCK, phosphoenolpyruvate carboxykinase (EC 4.1.1.49); PpsA, phosphoenolpyruvate synthase (EC 2.7.9.2); PykAF, pyruvate kinase (EC 2.7.1.40); AroFGH, deoxy-D-arabinoheptulosonate 7-phosphate synthase (EC 2.5.1.54); AroD, 3-dehydroquinate dehydratase (EC 4.2.1.10); AroL, Shikimate kinase II (EC 2.7.1.71); PheA, chorismate mutase/prephenate dehydratase (EC 5.4.99.5/4.2.1.51); TyrA, chorismate mutase/prephenate dehydrogenase (EC 5.4.99.5/1.3.1.12); TrpD, anthranilate phosphoribosyltransferase (EC 2.4.2.18); TrpE, anthranilate synthase component 1 (EC 4.1.3.27); TrpCF, multifunctional fusion protein (EC 4.1.1.48/5.3.1.24); TrpAB, tryptophan synthase (EC 4.2.1.20); PTL, phenol-tyrosine lyase from Pasteurella multocida (EC 4.1.99.2).

### Example 2: Targeted Removal of Proteins

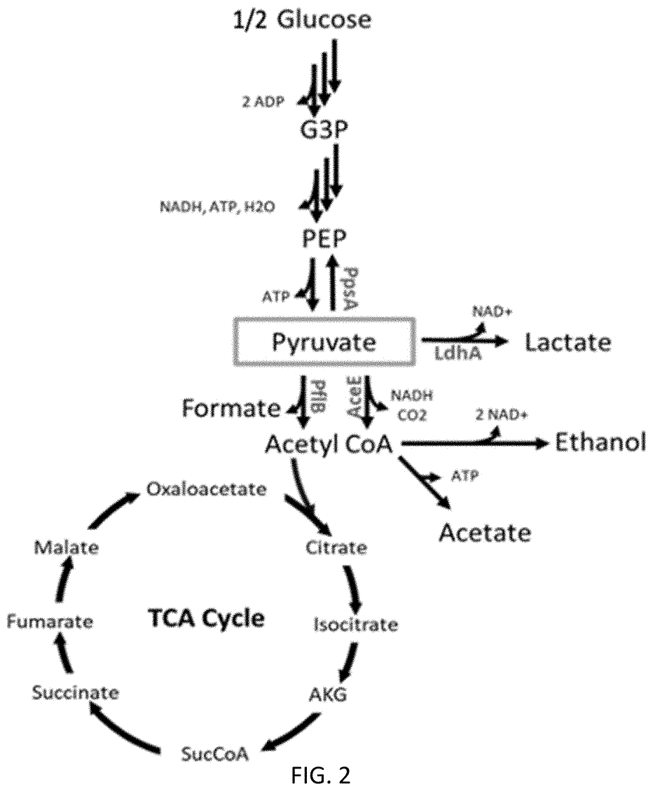

Pyruvate sits at the biochemical junction of glycolysis and the TCA cycle. It is a key intermediate in producing many food, cosmetic, pharmaceutical, and agricultural products whose improved production has been largely unexplored in cell-free systems. In order to create a pyruvate pooling phenotype in an E. coli cell-free extract, four proteins were chosen as targets for removal, LdhA, PflB, AceE, and PpsA (Table 1) (FIG. 2). These were chosen due to their direct role in consuming pyruvate as well as the likelihood that they are active in lysates. LdhA was selected as the production of lactate from pyruvate has been observed in cell-free systems generated under similar conditions to those prepared before (A. S. Karim, et al., Synth. Biol., vol. 5, no. 1, January 2020; Q. M. Dudley et al., Synth. Biol., vol. 4, no. 1, January 2019). Since LdhA has previously been reported to be absent in lysates derived from aerobically grown cultures, the inventors assumed that oxygen-limited zones are present in the cultures upon harvesting at mid-log phase (M. Bujara et al., Nat. Chem. Biol., vol. 7, no. 5, pp. 271-277, 2011). This is typical for cells grown in flasks, even under constant shaking, in media with high concentrations of glucose such as the 2×YPTG media (18 g L-1 glucose) (J. Soini, K. et al., Microb. Cell Fact., vol. 7, no. 1, p. 26, August 2008; S. O. Enfors et al., J. Biotechnol., vol. 85, no. 2, pp. 175-185, February 2001). Under this assumption, PflB is also likely expressed minimally in the culture and would be capable of carrying pyruvate flux for glycolytic fermentation. At the same time, pyruvate dehydrogenase (Pdh), responsible for pyruvate flux in aerobic conditions, is also expected to be expressed under these conditions as respiratory metabolism is reportedly active in S30 lysates. Pdh expressed in oxygen-limited cultures is additionally known to be functional in E. coli lysates as long as NADH concentrations are not allosterically inhibiting. During a cell-free metabolic reaction, one might expect PflB to be inactivated by oxygen due to its oxygen-sensitivity, and for pyruvate to acetyl-CoA flux to be controlled by Pdh. Such activity however has yet to be reported so both PflB and AceE, the E1 component of Pdh, were additionally chosen as targets here. The inventors also selected PpsA under the assumption that back-flux to phosphoenolpyruvate (PEP) might occur upon high pyruvate pooling in the lysates (FIG. 2). 6×His tags were fused on either the amino or carboxyl terminus by genetic modification based on an evaluation of literature related to previous purification attempts or crystal structures in order to find a non-inhibitory location (Table 4).

TABLE 4
Gene and protein information for MAGE targets
with a potential effect on pyruvate metabolism

| | | 6xHis-Tagged | MW |
|---|---|---|---|
| Gene | Protein | Terminus | (kDa) |
| pflB | Pyruvate formate-lyase | N-Terminal | 85 |
| IdhA | D-lactate dehydrogenase | N-Terminal | 36.53 |
| ppsA | Phosphoenolpyruyate | N-Terminal | 87.43 |
| | synthase | | |
| aceE | Pyruvate dehydrogenase | C-Terminal | 99.66 |
| | E1 | | |

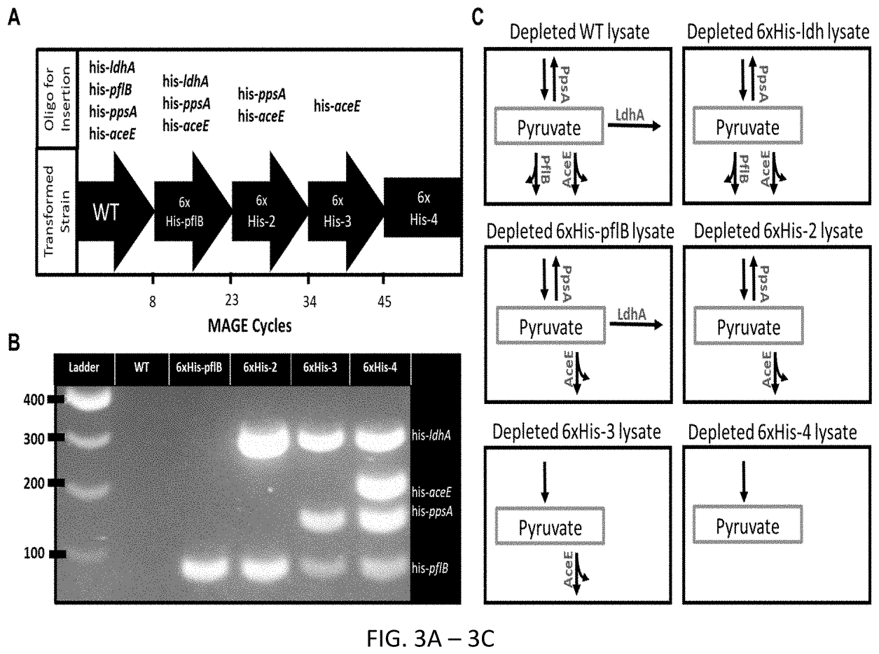

The pORTMAGE system was used instead of the traditional genome integrated system due to its potential transferability to multiple donor organisms including E. coli BL21 Star(DE3). Additionally, pORTMAGE is curable following genome engineering and relieves the metabolic burden on the cell that can be imparted due to plasmid maintenance. Colony screening was performed using MASC-PCR and further verified using Sanger sequencing. A total of 5 strains were used for this work. (Table 2). The strains included 6×His-pflB, 6×His-2 (6×His-pflB-ldhA), 6×His-3 (6×His-pflB-ldhA-ppsA), 6×His-4 (6×His-pflB-ldhA-ppsA-aceE) and 6×His-ldhA with each having a varying metabolic phenotype. 60 rounds of MAGE were needed to incorporate all four of the tags into the E. coli genome (FIG. 3 Top Panel) (Table 5). This is high when compared to studies producing single nucleotide changes but in line with other efforts using 6×His-tags with a genome incorporated MAGE system.

TABLE 5
Strains created for this study.

| Strain Name | Background | Modified Genes |
|---|---|---|
| 6xHis-pflb | BL21 Star (DE3) | 6xhis-pflB |
| 6xHis-ldh | BL21 Star (DE3) | 6xhis-ldhA |
| 6xHis-2 | BL21 Star (DE3) | 6xhis-ldhA, 6xhis-pflB |
| 6xHis-3 | BL21 Star (DE3) | 6xhis-idhA, 6xhis-pflB, 6xhis-ppsa |
| 6xHis-4 | BL21 Star (DE3) | 6xhis-ldhA, 6xhis-pflB, 6xhis-ppsa, 6xhis-aceE |

After curing each strain of the pORTMAGE plasmid, potential inhibitory effects on growth caused by the expression of tagged proteins were evaluated. Though the presence of the polyhistidine tags has previously been observed to cause growth defects due to the stability of tagged proteins, none of the cells produced for this work showed a significant drop in growth rate.

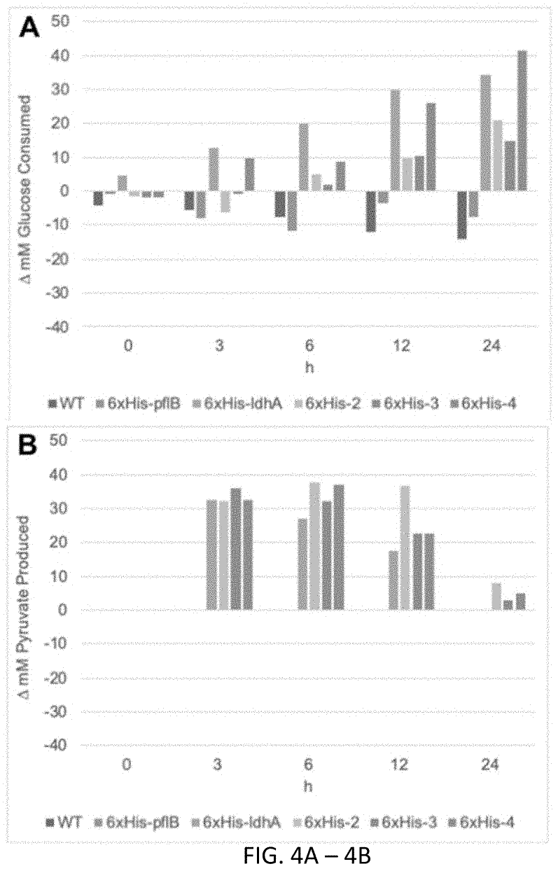

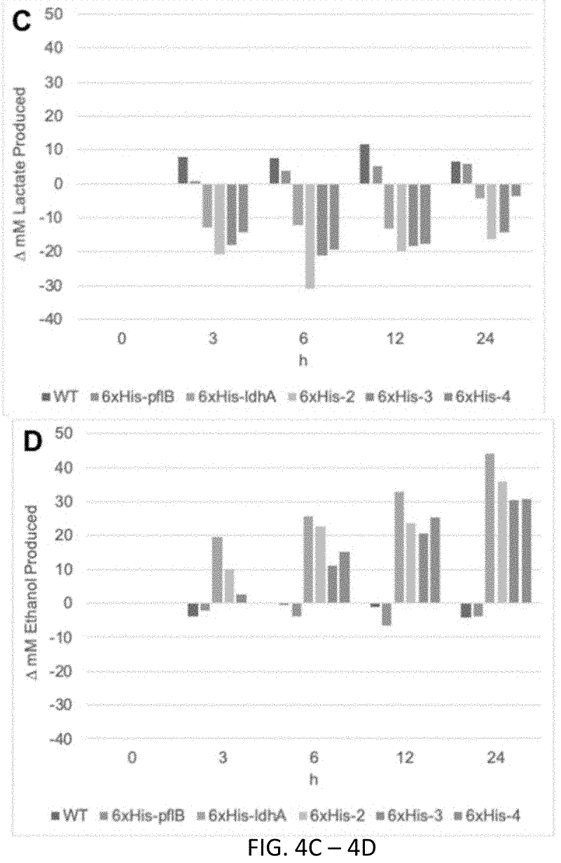

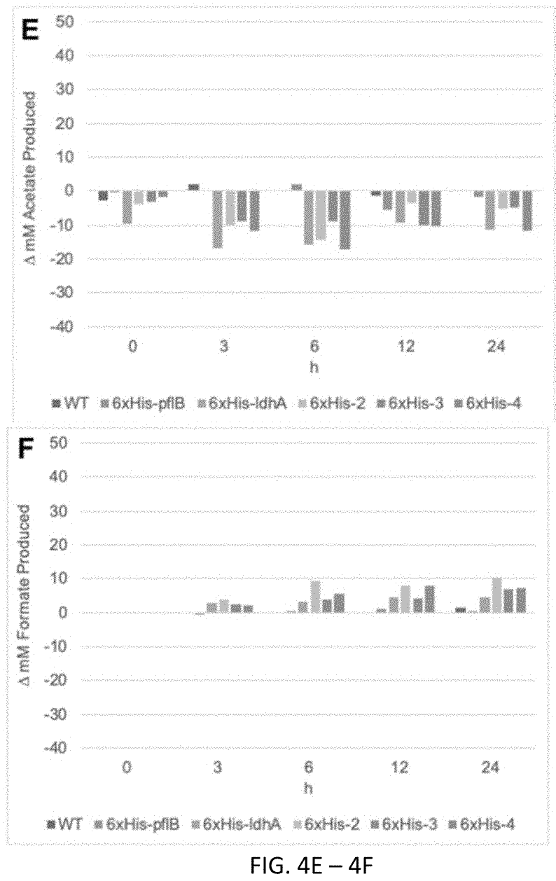

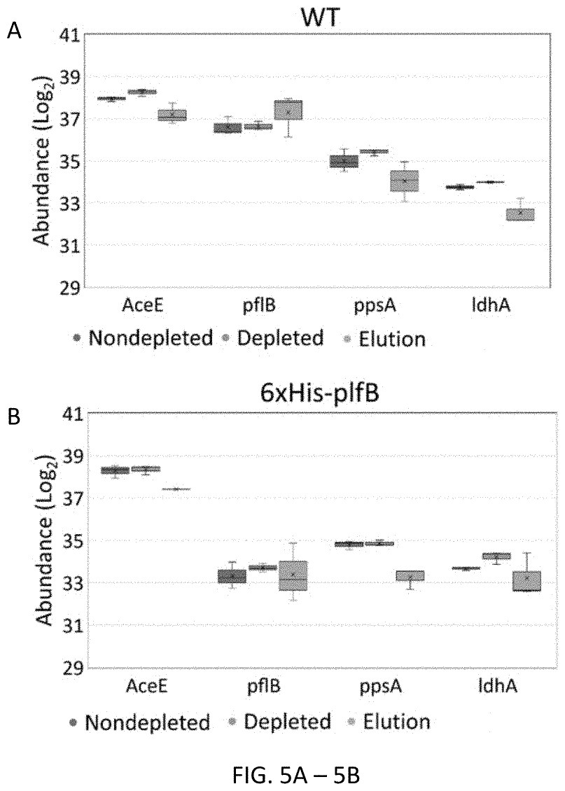

The effect of proteome reduction on the extract's metabolic profile was then tested by measurement of glucose consumption, pyruvate accumulation, and the pooling of fermentation end-products (i.e., lactate, ethanol, formate, and acetate) in a CFME reaction mix. As nonspecific binding is commonly associated with the use of 6×His-tags, the inventors evaluated whether the reduction method would result in significant alterations in lysate metabolism. Evidently, the wild-type derived lysate and the wild-type lysate taken through the depletion process have comparable glucose consumption and fermentation end-product pooling. Further, there is no apparent pyruvate accumulation after incubation of the WT lysates with cobalt beads, indicating that the depletion process does not remove proteins that affect cell-free pyruvate production in an appreciable manner Extracts derived from 6×His-pflB, 6×His-ldh, 6×His-2, 6×His-3, and 6×His-4 were thus reduced and assessed for glucose consumption and pyruvate build-up relative to their unreduced counterparts (FIGS. 4A and 4B). Significantly, none of the nondepleted extracts derived from these strains accumulated pyruvate, and metabolite profiles all trended similarly in terms of glucose consumption and fermentation end-product pooling. A noteworthy general observation from the metabolite profiles of the depleted 6×His-pflB, 6×His-ldh, 6×His-2, 6×His-3, and 6×His-4 lysates is that they all continue to accumulate downstream products of the target pyruvate-consuming enzymes, albeit with varying trends. This would suggest that the depletion method did not completely remove the targeted enzymes from the lysate proteome, but evidently allows a degree of targeted protein depletion that results in significant metabolic changes.

The targeted depletion of PflB from the 6×His-pflB extract results in a metabolic profile that is similar to its control counterpart in that neither accumulate pyruvate (FIG. 4B). Changes in glucose consumption and lactate, ethanol, acetate, and formate production between the depleted 6×His-pflB and WT lysates, relative to their nondepleted controls, are also insignificant (FIGS. 4A, 4C-4F). This metabolic phenotype could be expected considering that Pdh activity is active in crude extracts of E. coli. The Pdh complex has a higher affinity for pyruvate than PflB (Kₘ=0.4 versus 2.0 mM, respectively) when its activity is not inhibited by NADH such as in the presence of an NADH sink like LdhA (FIG. 2). Thus, regardless of whether PflB concentrations are decreased, Pdh could likely be an active route for flux towards ethanol and acetate in this lysate.

The lysate with targeted depletions of both PflB and LdhA (6×His-2, depleted) pooled 32 mM pyruvate relative to its nondepleted control in 3 h (FIG. 4B). This lysate additionally consumed glucose steadily while maintaining a >30 mM pyruvate concentration until 12 h. The depletion of LdhA is supported by the observation of a lower lactate concentration in reactions run in these lysates compared to their nondepleted counterparts (FIG. 4C). The rapid build-up of ethanol (20 mM in 3 h relative to the control) in these lysates, and the likely increased activation of aldehyde-alcohol dehydrogenase (AdhE) as an alternative sink for NAD+ regeneration, also supports successful LdhA depletion (FIG. 2, FIG. 4D). Acetate production is also not observed after LdhA depletion, presumably pointing to the increased funneling of acetyl-CoA from the Pdh reaction towards ethanol production to meet redox balance (FIG. 4E). Rather, acetate seems to be increasingly consumed as a secondary carbon source likely for generating more acetyl-CoA through the acetyl-CoA synthetase route (FIG. 4E). On the other hand, the contribution of depleted PflB to the observed metabolic phenotype in reactions run in this 6×His-2 lysate is not as immediately observable. However, at time points before 12 hours, there is a notable decrease in lactate and increased maintenance of high pyruvate concentrations (FIGS. 4B and 4C). In comparison, the depleted 6×His-ldhA lysate was not as efficient at retaining high pyruvate concentrations as the depleted 6×His-2 lysate (FIG. 4B). The depletion of both LdhA and PflB from the 6×His-2 extract may funnel pyruvate flux through Pdh, a claim bolstered by previous work showing NADH-insensitive Pdh to limit glucose fermentation in the absence of both PflB and LdhA. Thus, the pyruvate pooled up to 12 h in the depleted 6×His-2 lysate likely results from lowered glycolytic rates in these extracts. This interpretation is supported by the lowered consumption of glucose in the 6×His-2 lysate compared to the 6×His-ldhA lysate (FIG. 4A). The concomitant increase in ethanol production and significantly lowered lactate synthesis at 3 h and 6 h in the depleted lysate relative to its control additionally suggests that pyruvate flux is directed through Pdh-AdhE maintaining redox balance by generating net 1 mol NAD+ per mol pyruvate (FIGS. 4C and 4D). Compared to reactions run when depleting PflB and LdhA individually, the co-depletion of LdhA and PflB has an additive effect on cell-free pyruvate pooling. This interpretation suggests that oxygen-sensitive PflB is indeed active in E. coli crude extracts, which is supported by the observable production of formate after LdhA reductions (FIG. 4F). The inventors reason that decreasing the concentrations of the NAD regenerating LdhA enzyme limits the in vitro activity of formate dehydrogenases that require NAD as a substrate to decompose formate to CO₂ and H₂O, thus resulting in formate build-up.

Compared to the depleted 6×His-2 lysate, the pull-down of PpsA from the 6×His-3 lysate led to a steady decrease of the pyruvate concentration after 3 h (FIG. 4B). This observation presumably points to the importance of PpsA as an ATP sink in in vitro metabolism. E. coli glycolytic flux is naturally responsive to the cell's energy charge via the allosteric inhibition of phosphofructokinase and pyruvate kinase by ATP. The build-up of ATP in the depleted 6×His-3 lysate that results from glycolysis can lead to lower pyruvate production from glucose at later timepoints, a claim additionally supported by the decrease in relative glucose consumed at 24 h between the 6×His-2 and 6×His-3 lysates (FIG. 4A). The inventors additionally observed lower productivities (15 mM in 3 h) and final titers (41 mM) of ethanol formation in the depleted 6×His-3 extract compared to reactions run in the depleted 6×His-2 lysate (productivity=31 mM in 3 h; titer=55 mM) (FIG. 4D). The low initial accumulation of ethanol despite high pyruvate pooling from LdhA depletion is possibly due to decreased Pdh activity under high adenylate charge. The same condition can explain lowered ethanol production in the depleted 6×His-3 extract compared to the depleted 6×His-2 lysate (FIG. 4D). Alternatively, the less efficient ethanol pooling can be due to lowered synthesis rates of NADH from glycolysis after PpsA pull-down.

The targeted depletion of AceE, a component of Pdh, did not increase pyruvate pooling capabilities but led to the highest consumption of glucose observed (FIG. 4A). The inventors reason that perturbing the redox balance in the lysate through the pull-down of AceE and thus the depletion of NAD⁺-dependent Pdh activity increased the availability of NAD for increased glycolytic flux (FIG. 2). Moreover, the depletion of Pdh activity seems to shift the maintenance of redox balance back to LdhA at later timepoints, as suggested by the steady increase in lactate levels with the decrease in glucose stores (FIGS. 4A and 4C). NAD is thus continually regenerated by remaining LdhA and ethanol production for the NADH generating step in glycolysis, but this could possibly result in the build-up of glycolytic intermediates since the total consumption of 100 mM glucose is not fully accounted for by the concentrations of pooled fermentation end-products. In general, the rapid consumption of the NAD supply could be limiting due to the potential lack of cofactor recycling initiated by the decrease of LdhA levels. Pyruvate consumption experiments performed with the WT and 6×His-4 lysates show that a significant portion of the pyruvate consuming pathways remain robust after reduction evidencing that the constraint may be due to bottlenecks in upstream glycolysis and further shows that a balance between glucose and pyruvate consumption leads to the engineered pyruvate pooling phenotype (FIG. 4B).

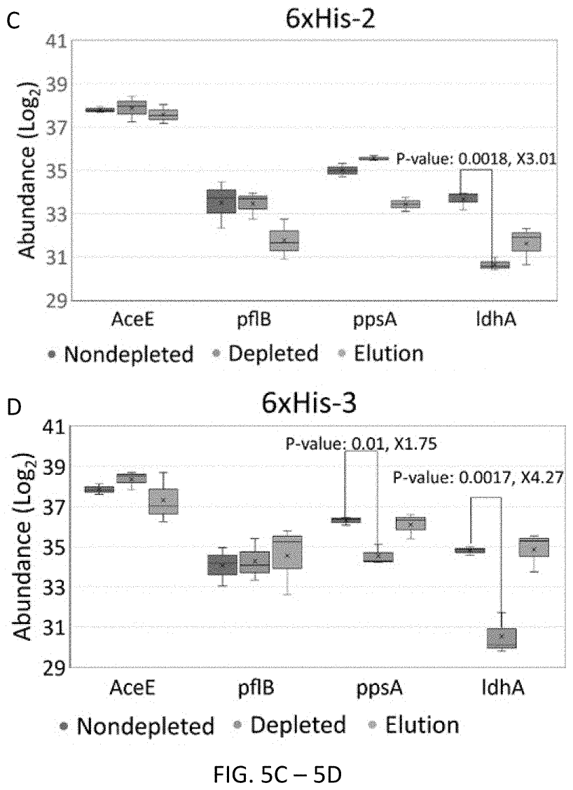

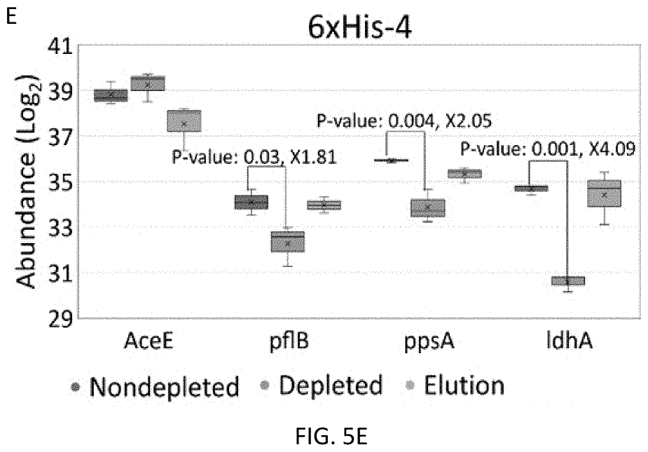

From the mass spectrometry-based proteomics profiling, it is evident that 6×His-tagged LdhA and PpsA could be removed from lysates, while significant removal of 6×His-tagged PflB was not successfully detected (FIGS. 5B to 5E). Although the decrease in PflB levels between the nondepleted and depleted 6×His-4 lysates met the significance threshold (pval<0.05), the change was only a 1.81-fold reduction compared to the significant decreases in LdhA and PpsA following lysate depletion (FIG. 5E). AceE was not observed to be pulled down after the purification of 6×His-tagged proteins from the 6×His-4 lysate. These findings are inconsistent with metabolic output data as the depletion of 6×His-4 lysate causes a more significant glucose consumption phenotype than extracts with fewer tags (FIG. 4A). However, anti-6×His western blots of eluants from the cobalt beads used to deplete the engineered extracts show an enrichment for each of the targeted proteins in their respective strains while no enrichment was seen in the elution from the WT. The corroborating evidence of the targeted metabolite analyses combined with antibody tagging leads us to conclude that the targeted proteins are being sufficiently removed and affect the reactivity of the extracts. The inconsistency in the results obtained from mass spectrometry and western blot analyses can be explained away as differences between the analytical techniques. Mass spectrometry is recognized for its ability to identify and quantify proteins in complex sample mixtures and for doing so with higher reliability, reproducibility, specificity, and sensitivity when compared to western blots. Here, the comprehensive, MS-based proteomic analyses involve different sample types, the nondepleted lysates, depleted lysates, and the eluants, and the different background signals may complicate comparisons. In contrast, the western blot experiment focuses on the analysis of a specific protein in the eluant protein fraction. These techniques complement each other and highlight the different strengths of the two approaches. Whereas the western blot provides confirmation of protein removal in the relatively simple eluant, it lacks the quantitative rigor of mass spectrometry that is needed for comprehensive analyses of complex samples. Therefore, the western blot provides an orthogonal complement to the MS-based results and provides support for the observed metabolic output data. While western blot analysis validated the depletion of proteins not identified through comparative mass spectrometry analysis, future efforts can benefit from a more targeted proteomics approach using labeled peptides to determine absolute quantitative measurements of the method's depletion efficiency for targeted proteins.

Nonetheless, the comparative mass spectrometry analysis provided additional information about the method described herein. The results show that the incorporation of 6×His-tags into the genomes had minimal effects on the expression of pyruvate-consuming enzymes in all strains' proteomes (FIGS. 5A-5E, blue boxes), allowing the pull-down of one target protein without altering the concentrations of other pyruvate-consuming enzymes. This observation is corroborated by the comparable trends among the metabolite profiles of all nondepleted lysates. This advances the method for precisely generating unconventional metabolic phenotypes that cannot be achieved via gene deletions, since knock-outs of metabolic genes would incite global proteomic and thus metabolic changes in cells. The inventors further analyzed relative proteomic changes in the nondepleted and depleted extracts to determine whether the method resulted in the removal of off-target proteins. Importantly, the process of depleting the proteome did not seem to significantly impact proteins in central metabolism outside of those targeted by the tagging process. When comparing the depleted and nondepleted WT lysates, in the 58 proteins with a greater than 4-fold reduction, none were connected to central metabolism outside of roles in membrane transport. Future efforts will seek to minimize off-target effects in order to improve the general applicability of lysate proteome engineering. Though outside the initial scope of this study as the main prospect was to show the use of enzyme depletion as a tool for CFME, targeted proteomics could be an effective tool to connect concentrations of depleted proteins with their resultant metabolic profile.

Targeted depletion of a lysate proteome enables a rapid means to manipulate central metabolism without the possible drawback of cultivating “sick” organisms as often results from traditional, in vivo metabolic engineering efforts. The pORTMAGE system offers the potential for extension of this engineering strategy to other, non-model cell-free chassis. Though not all proteins targeted for depletion could be shown to be depleted in substantial quantities through proteomics, the analysis of the metabolic products and western blot analysis shows clear differences between the extracts following each tagging and only following depletion. In contrast with gene knockout strategies that result in global proteomic changes during source strain cultivation, this method allows removal of selected proteins from a lysate proteomic background that is similar to the wild type derived extracts, allowing targeted manipulation of lysate proteomes. Thus, although lysates derived from the deletion of a target gene or the post-lysis depletion of its corresponding protein are expected to have different metabolic phenotypes, the instant CFME approach could be broadly applied to yield metabolic states that are not traditionally possible in living organisms. Future improvements to lysate proteome engineering could make use of multiplex genome engineering methods that are amenable to the insertion of larger tags as MAGE based methods are naturally limited to low-base pair insertions. To further advance the depletion of specific proteins in the lysate's proteome, orthogonal protein degradation systems could be employed wherein proteins are genomically tagged and degraded in a cell-free extract using an exogenous protease. The mf-lon protease system serves this function through a 27 amino acid long peptide and could allow for titration experiments leading to complete degradation of the proteins of interest. A key factor to note stems from MAGE's limited throughput when making large additions to the genome. Whereas single base changes can be added with ease, longer tags such as 6×His tags, are near the edge of feasibility for MAGE tagging. Organisms such as Vibrio natriegens can take advantage of a MAGE like process termed MUGENT that allows for significantly longer incorporations at the cost of using a donor strain with less study than E. coli.

Shown in this disclosure is the use of genome engineering to create protein modifications that allow for the control of metabolic activity in cellular lysates. This cell-free metabolic engineering strategy allows for the targeted removal of enzymes that can enable the focused production of metabolites from simple precursors using rapidly prepared crude extracts that would otherwise lead to changes in metabolic state and significant growth defects in living cells. The ability to extract pyruvate degrading enzymes, leading to unconventional metabolic states, was engineered and shown to be capable of pooling pyruvate for a significant period of time as well as improving the ethanol titer of the extract. The ability to direct metabolic flux in cell-free systems and create proteomes untenable to living cells was demonstrated. The flexibility of CFME systems highlights the significant value they hold as novel bioproduction platforms. The advances made in this work can be extended to design molecule specific donor strains for natural product biosynthesis, such as for polyketides or carbohydrates, through the removal of defined inhibitory reactions. The removal of specific components of crude lysates allows for more complex reaction networks to be employed in the development of CFME bioproduction platforms. As CFME begins to tackle new challenges related to antibiotic, fuel, and, materials production, innovative engineering tools and techniques designed to improve its efficiency will be crucial to advancing the scope and adoption of cell-free biological production.

### Example 3: Targeted Growth Medium Dropouts Promote Aromatic Compound Synthesis in Crude E. coli Cell-Free Systems

Progress in cell-free protein synthesis (CFPS) has spurred resurgent interest in engineering complex biological metabolism outside of the cell. Unlike purified enzyme systems, crude cell-free systems can be prepared for a fraction of the cost and contain endogenous cellular pathways that can be activated for biosynthesis. Endogenous activity performs essential functions in cell-free systems including substrate biosynthesis and energy regeneration; however, use of crude cell-free systems for bioproduction has been hampered by the under-described complexity of the metabolic networks inherent to a crude lysate. Physical and chemical cultivation parameters influence the endogenous activity of the resulting lysate, but targeted efforts to engineer this activity by manipulation of these non-genetic factors has been limited. Here, growth medium composition was manipulated to improve the one-pot in vitro biosynthesis of phenol from glucose via the expression of Pasteurella multocida phenol-tyrosine lyase in crude E. coli lysates. Crude cell lysate metabolic activity was focused towards the limiting precursor tyrosine by targeted growth medium dropouts guided by proteomics. The result is the activation of a 25-step enzymatic reaction cascade involving at least three endogenous E. coli metabolic pathways. Additional modification of this system, through CFPS of feedback intolerant AroG improves yield. This effort demonstrates the ability to activate a long, complex pathway in vitro and provides a framework for harnessing the metabolic potential of diverse organisms for cell-free metabolic engineering. The more than six-fold increase in phenol yield with limited genetic manipulation demonstrates the benefits of optimizing growth medium for crude cell-free extract production and illustrates the advantages of a systems approach to cell-free metabolic engineering.

Enabling Phenol Production in E. coli Cell-Free Systems

Aromatic compounds are valuable chemicals with uses as industrial solvents, fuels, and substrates for chemical synthesis. Largely derived from petroleum, manufacturing of aromatic compounds by microbial fermentation of a low-cost sugar substrate would present an environmentally friendly alternative. As aromatic rings are present in nucleotide bases and in three of the proteinogenic amino acids, many organisms have biosynthetic pathways to produce aromatic compounds. The building blocks for the aromatic amino acids phenylalanine, tryptophan, and tyrosine result from the shikimate pathway. Additionally, the shikimate pathway is the metabolic launching point for biosynthesis of phenylpropanoids, a diverse class of secondary metabolites synthesized from iterative additions of malonyl- and coumaroyl-CoAs, that include medicinally valuable compounds such as flavonoids and stilbenoids. Others have succeeded in developing in vitro biosynthetic pathways for highly conjugated compounds including acyl-CoAs, but production of aromatic compounds by the shikimate pathway in vitro has not been explored.

Phenol is one of the simplest aromatic compounds, consisting of a six-carbon aromatic ring appended with a single hydroxyl group. Phenol-tyrosine lyases (PTL, 4.1.99.2) from various enterobacteria have been found to catalyze the synthesis of phenol from the amino acid tyrosine. Improving substrate availability by engineering tyrosine biosynthesis increased phenol yield, but cytotoxicity limited productivity. The reduced impact of highly cytotoxic products on cell-free bioproduction platforms provides an attractive alternative for phenol biosynthesis.

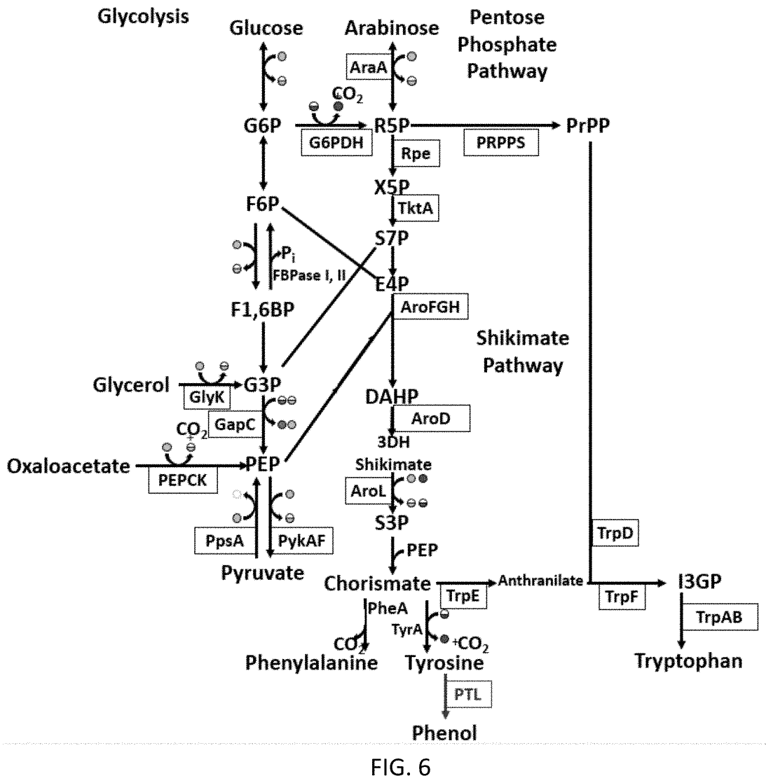

While many microorganisms, including E. coli, can make their own tyrosine, high-yield tyrosine biosynthesis is a complex phenotype. Tyrosine biosynthesis requires not only the four and three carbon building blocks, erythrose 4-phosphate (E4P) and phosphoenolpyruvate (PEP), which are condensed to form 3-deoxy-D-arabino-heptuloseonate 7-phosphate (DAHP), but an additional PEP, ATP, and NADPH are also required. NADPH can be regenerated through the prephenate dehydrogenase activity of TyrA (5.4.99.5/1.3.1.12), however PEP and ATP must be generated outside of the shikimate pathway (FIG. 6).

In this work, the one-pot in vitro biosynthesis of phenol was achieved by coupling endogenous production of tyrosine from glucose with CFPS of PTL from Pasteurella multocida. Fully-labeled ¹³C₆ glucose was used as the carbon source to distinguish between phenol synthesized from amino acids added as a substrate for CFPS and the desired full pathway. Glucose is rapidly converted into acetate and lactate in crude E. coli lysate lowering the reaction pH; to counteract this, a buffer with a lower pH range, Bis-Tris, was used in lieu of the commonly used HEPES buffer. CFPS and phenol production both require exogenous ATP; as oxidative phosphorylation is not expected to be active in systems lysed by sonication, creatine phosphate and creatine kinase were added to these reactions. Reactions were also supplemented with exogenous PEP as an additional PEP molecule is required to synthesize chorismate; this molecule is released as pyruvate upon generation of tyrosine by PTL. Simultaneous addition of PTL template DNA, labeled glucose, and creatine kinase initiated in vitro phenol production, which proceeded over the course of 48 hours and was quantified by GC/MS. Recent work has shown that exogenous tRNAs are not necessary to facilitate CFPS in crude E. coli lysates and were not included in the reaction mixtures.

Characterization of Crude Cell Free Systems Prepared from Defined Media

While variables including aeration and growth temperature also impact this activity, the removal of critical metabolites from the growth medium can facilitate targeted activation of biosynthetic pathways for these metabolites in vivo and increased abundance of pathway enzymes in the resulting crude lysates. Small changes in available nutrients and growth conditions result in large compensatory shifts in protein abundance which can be observed with shotgun proteomics. To provide fine control over medium conditions, a cell-free system based upon growth on defined media was developed. Using this system, variables potentially impacting tyrosine production including carbon source and presence of aromatic compounds in the medium were investigated. In particular, the effects of aromatic amino acids and nucleotide bases in the medium were explored. Impacts of each change to the growth medium were evaluated by shotgun proteomics and used to inform subsequent modifications. While variables including aeration and growth temperature also impact this activity, the removal of critical metabolites from the growth medium can facilitate targeted activation of biosynthetic pathways for these metabolites in vivo and increased abundance of pathway enzymes in the resulting crude lysates. Small changes in available nutrients and growth conditions result in large compensatory shifts in protein abundance which can be observed with shotgun proteomics. To provide fine control over medium conditions, a cell-free system based upon growth on defined media was developed. Using this system, variables potentially impacting tyrosine production including carbon source and presence of aromatic compounds in the medium were investigated. In particular, the effects of aromatic amino acids and nucleotide bases in the medium were explored. Impacts of each change to the growth medium were evaluated by shotgun proteomics and used to inform subsequent modifications. All media compositions are detailed in Table 6.

TABLE 6
Composition of each EZ Rich derived media.

| Growth Condition | Supplement EZ | ACGU mix | Carbon Source |
|---|---|---|---|
| EZ Rich | + | + | 11 mM Glucose |
| EzGlc | + | + | 100 mM Glucose |
| AAA | −Trp, −Tyr, −Phe | + | 100 mM Glucose |
| ACGU | + | − | 100 mM Educose |
| EzAra | + | + | 100 mM Arabinose |
| EzG1y | + | + | 100 mM Glycerol |
| DDGlc | −Trp, −Tyr, −Phe | − | 100 mM Glucose |

E. coli cell-free systems for protein production are generally grown using the rich, complex medium YTPG, which consists of five components: yeast extract, tryptone, NaCl, potassium phosphate and glucose. Yeast extract and tryptone contain many different complex biomolecules with significant batch to batch variations; this presents limited opportunity for modification and optimization. The rich, defined medium described by Neidhardt et al. and commercially available as “EZ Rich” by Teknova provides greater flexibility as each component can be individually changed (Neidhardt, F. C. et al., “Culture medium for enterobacteria.” Journal of Bacteriology 119.3 (1974): 736-747.). A modified CFPS extract preparation protocol was developed based upon EZ Rich medium.

TABLE 7
Comparison of amino acid concentrations
in YTPG and EZ Rich media.

| Amino | YTPG conc | 1 × EZ ich | % of YTPG |
|---|---|---|---|
| acid | (mM) | conc. (mM) | conc |
| Ala | 9.76 | 0.80 | 8 |
| Arg | 4.65 | 5.20 | 112 |
| Asp | 12.35 | 0.40 | 3 |
| Cys | 1.02 | 0.10 | 10 |
| Glu | 27.81 | 0.60 | 2 |
| Gly | 7.15 | 0.80 | 11 |
| His | 3.07 | 0.20 | 7 |
| Ile | 7.45 | 0.40 | 5 |
| Leu | 12.37 | 0.80 | 6 |
| Lys | 10.00 | 0.40 | 4 |
| Met | 3.06 | 0.20 | 7 |
| Phe | 5.47 | 0.40 | 7 |
| Pro | 13.97 | 0.40 | 3 |
| Ser | 10.30 | 10.00 | 97 |
| Thr | 7.43 | 0.40 | 5 |
| Trp | 1.21 | 0.10 | 8 |
| Tyr | 2.08 | 0.20 | 10 |
| Val | 10.17 | 0.60 | 6 |

Maintaining CFPS capabilities was a priority in the development of this system as in vitro protein expression can shorten design-build-test cycles and allow synthesis of different end products. Further, as has been demonstrated in the engineering of isoprenoid biosynthesis, tuning of expression levels of terminal synthases is an important step to optimize product yield. To develop a crude cell-free system grown from defined medium, the growth protocol for YTPG based cell-free systems was followed with modification. Optimal OD₆₀₀ at harvest was adjusted to compensate for a higher terminal OD₆₀₀ compared to YTPG. Cells grown in defined medium and YTPG were induced with IPTG at the same OD₆₀₀ (0.8); despite differences in terminal OD₆₀₀, no significant difference in T7 polymerase was detected across any lysate preparation in this work. Others have found that CFPS is possible in lysates harvested during stationary phase and suggest acetate accumulation in the medium reduces in vitro protein synthesis rates. Notably, EZ Rich derived media are buffered and may mitigate this effect.

The glucose concentration of the EZ Rich medium was adjusted to create media more comparable to YTPG. This adjusted medium, EzGlc, and its variants were used for all further investigation. CFPS yield of sfGFP from plasmid pJL1 was assessed for all cell-free systems generated from EzGlc variants for this study by relative fluorescence. Absolute quantitation of protein yield continues to be essential for optimizing CFPS systems; however, as phenol yield was the optimization target of this work a relative measure of CFPS yield was used to quickly assess changes between conditions. The rates of cell-free protein synthesis for all variants were greatest between 40 minutes and 80 minutes after the beginning of the reaction. Two variant systems, AAA and ACGU, were observed to have increased yields of sfGFP by CFPS. The AAA variant was observed to have the greatest protein synthesis rate; however, this high rate was not observed in the related DDGlc variant.

Cell-free protein synthesis is a complex process involving numerous enzymes. To assess the impact of the growth conditions on the proteins involved in CFPS, the 87 proteins in the minimal PURE system were identified, and statistical differences in their abundances were measured. Across cell-free systems generated for this study, 26 protein elements of the PURE system were identified to be differentially abundant with a fold change of greater than two compared to YTPG in at least one condition. It remains unclear which individual proteins have the largest impact on in vitro protein synthesis yield. However, others suggest that some variation in concentration of ribosome subunits is permissible, which is corroborated by these data.

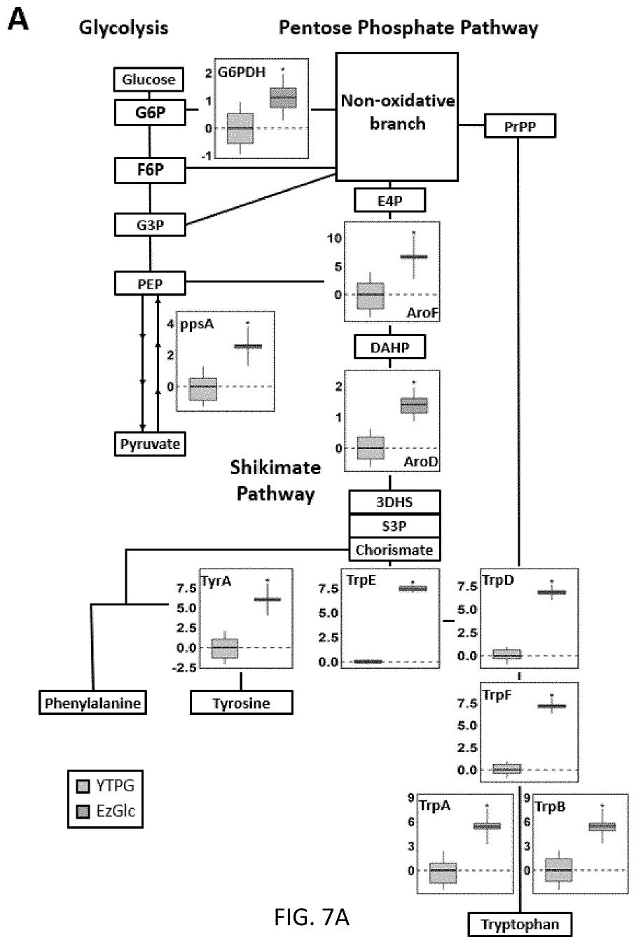

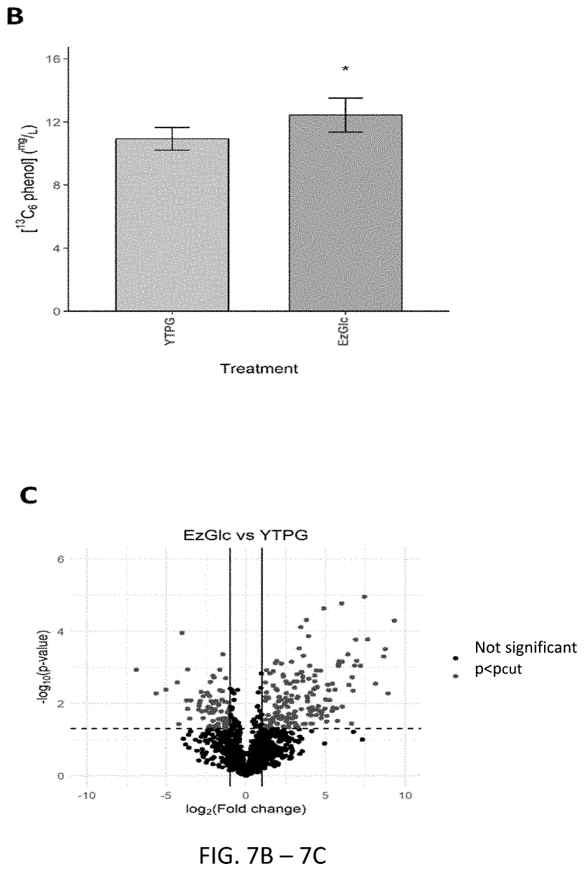

Cell-free phenol yield was assessed in both YTPG and EzGlc cell-free systems (FIGS. 7A-7C). Additionally, the protein content of each system was measured and compared with a focus on changes within the 25 enzymes directly involved in tyrosine biosynthesis (FIG. 7A). Notably, there was a large increase in nearly all amino acid biosynthesis pathways when cell-free systems are prepared from EzGlc medium compared to the YTPG extracts. This may result from the lower amino acid concentrations present in EzGlc. The impacted proteins include tyrosine biosynthesis enzymes DAHP synthase (AroF, 2.5.1.54), 3-dehydroquinate dehydratase (AroD, 4.2.1.10), and TyrA, which were increased by 98-fold, 2.5-fold and 66-fold, respectively. However, despite these large increases in protein abundance, phenol yield only increased from 10.9 mg/L in the YTPG condition to 12.4 mg/L in the EzGlc condition (p=0.048, FIG. 7B). This comparatively small increase in yield is likely caused by the addition of new carbon sinks resulting from an increased prevalence of other amino acid biosynthesis pathways.

### Impact of Carbon Source on In Vitro Phenol Biosynthesis

In E. coli, all three aromatic amino acids are derived from chorismate, the nine-carbon product of the shikimate pathway. Metabolic flux to each amino acid is regulated primarily by transcriptional control. While endogenous transcription, and the associated regulation, are not expected to be present in cell-free systems, tyrosine biosynthesis is also limited by the availability of shikimate pathway precursors PEP and E4P derived from glycolysis and the pentose phosphate pathway, respectively.

With the goal of increasing precursor supply, two media with alternative carbon sources were prepared. The EzAra medium contains the pentose sugar arabinose, which was hypothesized to upregulate transketolase and transaldolase as arabinose enters E. coli metabolism through the pentose phosphate pathway. Medium EzGly contains glycerol which is converted into the glycolytic intermediate 3-phosphoglycerate and was added to upregulate gluconeogenesis and stabilize the pool of PEP.

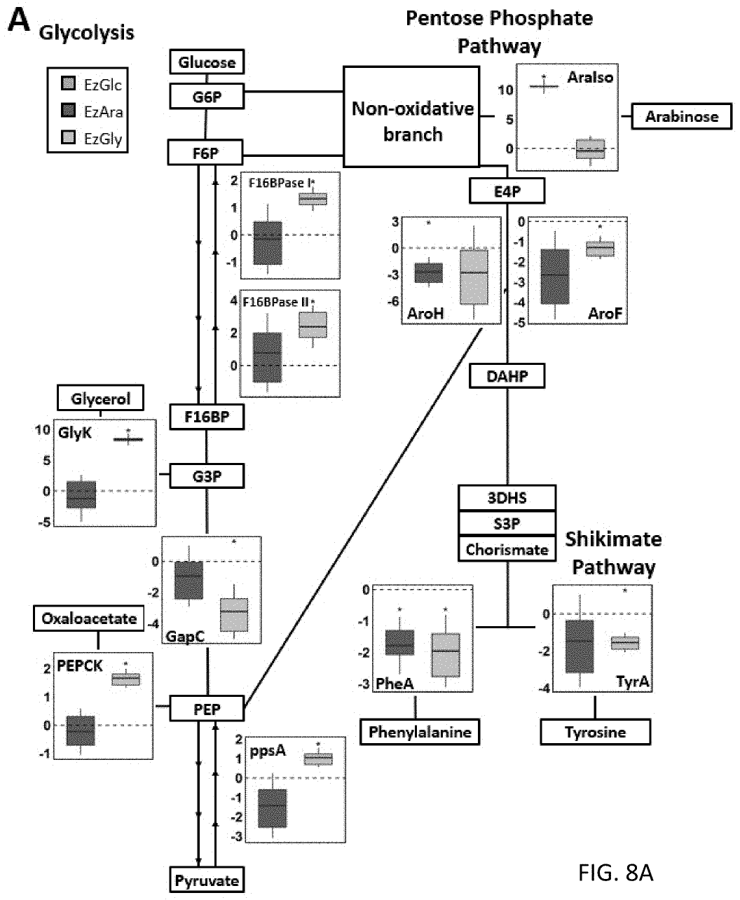

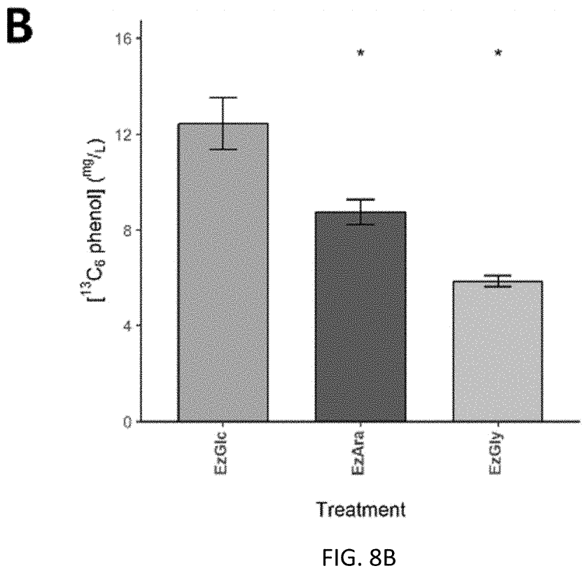

Changing carbon sources resulted in large increases in several proteins (FIGS. 8A-8B). Glycerol kinase (GlyK, 2.7.1.30) abundance was increased by 128-fold in the EzGly condition and arabinose isomerase (AraA, 5.3.1.4) was increased by three orders of magnitude in the EzAra condition. Changes within central carbon metabolism were less dramatic, but nonetheless significant. Decreased abundance of glyceraldehyde-3-phosphate dehydrogenase (GapC, 1.2.1.12) and increased abundance of both fructose 1,6, bisphosphatase I and II (FBPase I and II, 3.1.3.11) were observed in the EzGly conditions and may result in an increased gluconeogenic potential. Further, abundance of both phosphoenolpyruvate carboxykinase (PEPCK, 4.1.1.49) and phosphoenolpyruvate synthase (PpsA, 2.7.9.2) were increased by growth on EzGly (FIG. 8A). This suggests that growth on a triose has the potential to stabilize the pool of PEP in a cell lysate. The EzAra growth medium did not result in any other substantial changes within tyrosine biosynthesis.

Unfortunately, growth on media EzAra and EzGly resulted in decreasing two DAHP synthase isozymes (AroHF, 2.5.1.54), which would limit tyrosine production. Further, both conditions reduced abundance of TyrA, which in vivo engineering efforts have shown is critical to tyrosine production. Although both conditions resulted in the reduced abundance of the competing bifunctional phenylalanine biosynthesis enzyme PheA (5.4.99.5/4.1.1.51), it does not appear as though this compensated for the deleterious changes. The EzAra and EzGly cell-free systems both underperformed the EzGlc and base YTPG cell-free systems producing 8.8 mg/L and 5.8 mg/L phenol, respectively. Due to their reduced phenol yield and the lower abundance of key enzymes, both the EzAra and EzGly media were not studied further.

### Example 4: Removing Medium Components During Growth Activates Biosynthetic Pathways in Cell Lysates

Inventors observed that abundances of glycolytic enzymes were relatively unchanged across several growth conditions. However, larger shifts in protein abundance were observed outside of central carbon metabolism. With the goal of increasing the activity of aromatic compound biosynthesis in vitro, several dropout media were created. Medium AAA is a tyrosine, tryptophan and phenylalanine dropout that was hypothesized to increase flux towards the aromatic amino acids. Dropout medium AAA was prepared using a 5× EZ supplement from a second supplier (BioWorld), which may introduce variation in medium composition. Medium ACGU is a dropout of the EZ Rich nucleotide base mixture. As purine nucleotide bases are synthesized from ribose-5-phosphate, this dropout was expected to increase flux to the pentose phosphate pathway and increase yield of aromatic compounds in vitro. Ribose-5-phosphate is expected to be an important intermediate in lysates grown with and without the nucleotide base mixture as it forms the sugar backbone of nucleic acids.

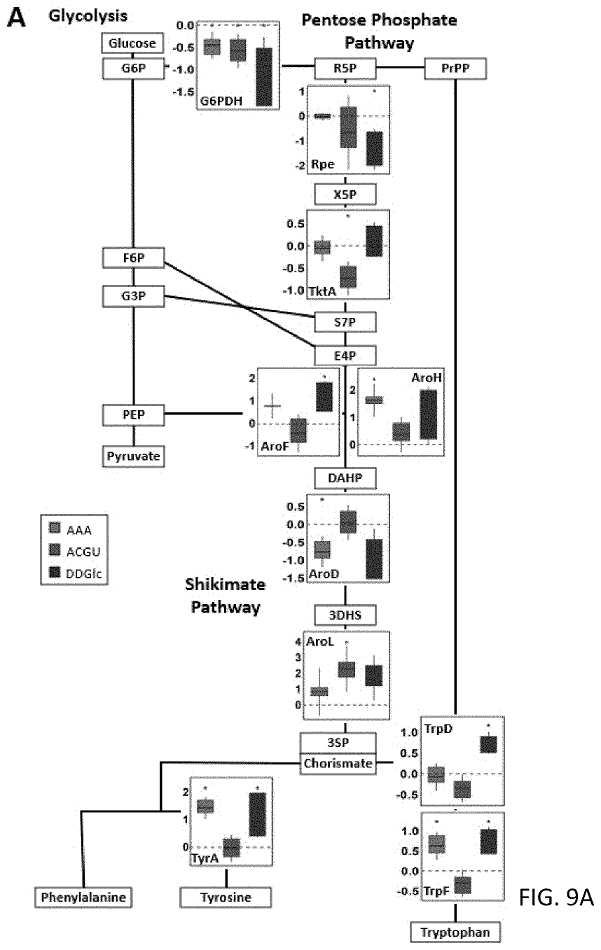

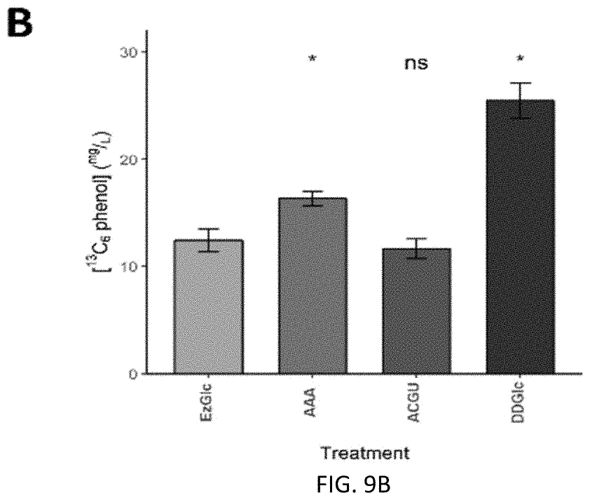

Medium AAA performed as predicted with increases in rate-limiting DAHP synthases AroH and AroF as well as tyrosine-forming dehydrogenase TyrA. However, 3-dehydroquinate synthetase (AroD, 4.2.3.4) abundance was reduced by nearly two-fold and enzymes known to impact E4P supply were not affected (FIG. 9A). The impact of medium ACGU was less predictable. An absence of nucleotide bases reduced the abundance of both oxidative pentose phosphate pathway enzyme glucose-6-phosphate dehydrogenase (G6PDH, 1.1.1.49) and transketolase (TktA, 2.2.1.1). Intriguingly, growth on medium ACGU increased the abundance of shikimate kinase 2 (AroL, 2.7.1.71) four-fold. This effect may be elicited by the increased demand for tetrahydrofolic acid, a chorismate derivative and an essential cofactor in nucleic acid biosynthesis.

Though decreases in AroD in the AAA condition were observed, the increases in rate-limiting enzymes resulted in a 31.6% (p<0.05) increase in phenol yield to 16.4 mg/L (FIG. 9B). Further, cell extracts prepared without nucleotide bases synthesized phenol at amounts similar to EzGlc despite the reduced abundance of pentose phosphate pathway enzymes. Through evaluation of individual changes to growth medium composition by both their impact on phenol yield and the changes they elicit in the lysate proteome, new composite growth conditions can be designed to target specific metabolic pathways.

While the AAA medium was the only one able to increase in vitro phenol yield, growth on the ACGU medium led to increased abundance of unexpected enzymes within the shikimate pathway, which provoked further investigation. A medium dropping out both aromatic amino acids and nucleotide bases with glucose as the carbon source was explored to combine the positive effects of these two sets of changes to the growth medium composition. This medium, dubbed double dropout glucose (DDGlc), was used to prepare a cell-free system and characterized as previously described. This new system further improved phenol biosynthesis to 25.8 mg/L, a 104.8% increase compared to EzGlc (p<0.05) and increased the abundance of several unique enzymes.

The extract derived from the DDGlc medium shares many of the proteins of increased abundance found in its parent cell-free systems, AAA and ACGU. TyrA, AroH and AroL all show increased abundance compared to the EzGlc cell-free system. While the abundance of 3-dehydroquinate synthase is still reduced in the DDGlc cell-free system, the reduction of transketolase abundance in the ACGU condition is not maintained in the double dropout. As there are many potential sinks of PEP, determination of the metabolic fate of PEP in the various cell-free systems will likely be necessary to further increase phenol yield.

The double dropout medium results in the unique reduction of the abundance of ribulose 5-phosphate epimerase (Rpe, 5.1.3.1), which was not observed in any of the parent conditions. This change has the potential to impact E4P supply by limiting the amount of glucose which enters the pentose phosphate pathway in vitro. Further, the DDGlc medium increased the abundance of anthranilate PrPP transferase (TrpD, 2.4.2.18), a key enzyme in tryptophan biosynthesis which utilizes resources from both the shikimate and pentose phosphate pathway. It is possible that the observed increased flux to tyrosine is a consequence of a greater increase in flux to tryptophan. Eliminating the conversion of chorismate to anthranilate would channel shikimate pathway products towards tyrosine.

### CFPS of the Rate-Limiting Enzyme AroG

Post-lysis addition of enzymes by cell-free protein synthesis not only enables synthesis of heterologous products but can also facilitate engineering of endogenous metabolism through expression of these bottleneck enzymes and their variants. Limitations on phenol yield by both substrate availability and CFPS yield of PTL were investigated. To investigate limitations on tyrosine availability, potential bottleneck enzymes were identified from proteomics data and co-expressed with PTL in vitro in the DDGlc system.

In the two media with elevated in vitro ¹³C₆ phenol yields, DAHP synthases were among the most highly increased enzymes in the tyrosine biosynthesis pathway. Expression of additional copies of endogenous rate-limiting enzymes can improve flux towards specific pathways to overcome bottlenecks³⁴. Expression of multiple constructs in a single cell-free reaction may reduce individual enzyme expression levels through competition for resources; however, total in vitro protein synthesis yield is only mildly affected³⁵. To control for influences of CFPS yield, a fixed DNA template concentration of 10 ng/μL was divided evenly between PTL and the co-expressed enzyme; co-expression of a metabolically inactive protein, sfGFP, resulted in an expected reduction of both labeled and unlabeled phenol yield due to reduction of PTL template concentration. CFPS of both PTL and DAHP synthase AroG in the DDGlc lysate increases ¹³C₆ phenol yield by 80.5% when compared to the control co-expression. This co-expression also increases unlabeled phenol yield by 61.1%, representing a general widening of the bottleneck into the shikimate pathway.

Increasing the CFPS yield of crude E. coli systems has been of much interest in recent years and is crucial to CFME efforts; changes to both growth protocol and medium formulation have been shown to have an impact on CFPS yield. Three media, AAA, ACGU, and EzAra, were shown to increase CFPS yield compared to EzGlc by 58.6%, 31% and 14.5%, respectively; however, these increases are not well correlated with increased phenol yield. Of the systems with increased CFPS yield, only one, AAA, also had increased ¹³C₆ phenol yield. ACGU did not show increased labeled or unlabeled phenol yield and ¹³C₆ phenol yield was reduced by 29.6% (p<0.05) in EzAra. Furthermore, the system with the highest yield of ¹³C₆ and unlabeled phenol, DDGlc, did not show an increase in CFPS yield compared to EzGlc.

Improvement in CFPS yield, through lysate modification or increased template concentration could improve phenol yield, but PTL activity has not been observed to be limiting to in vitro phenol biosynthesis below tyrosine concentrations approaching 1 mM. As determined by proteomics of a trypsin digest of a single in vitro phenol biosynthesis reaction prepared from medium DDGlc, the measured abundance and coverage of PTL derived peptides, synthesized in vitro, are similar to those of endogenous proteins in the lysate. Intriguingly, co-expression of AroG alongside PTL, each at 5 ng/μL, resulted in similar ¹³C₆ phenol yield as expression of PTL alone at 10 ng/μL (p=0.11). However, the co-expression also resulted in a 33% decrease in unlabeled phenol yield. The relationship between PTL template and unlabeled phenol production suggests that there are abundant unlabeled phenol precursors in the lysate, including the 2 mM tyrosine added for CFPS. However, the increase in fully labeled phenol with the co-expression of AroG implies that while PTL abundance, and by extension CFPS yield, impacts phenol yield, upstream enzyme abundance and activity drives ¹³C₆ phenol yield in this system.

In addition to synthesizing additional copies of endogenous enzymes, mutants can be expressed to overcome regulation. Three isozymes of DAHP synthase, AroGHF, carry out the rate-limiting condensation of E4P and PEP in aromatic amino acid biosynthesis; each isozyme is allosterically inhibited by one of the aromatic amino acids. AroG is sensitive to feedback inhibition by phenylalanine and makes up 80% of endogenous DAHP synthase activity. However, a single amino acid mutation (146D->N) in AroG abolishes feedback inhibition³⁹. CFPS of this feedback insensitive mutant along with PTL resulted in an improved ¹³C₆ phenol yield of 67.1 mg/L, representing a 440% increase compared to the control co-expression. Intriguingly, unlabeled phenol yield is not significantly changed between the feedback sensitive and insensitive co-expression, suggesting most of the unlabeled phenol is being synthesized from shikimate pathway intermediates present during lysis or tyrosine added for CFPS. While simultaneous expression of feedback insensitive AroG and PTL resulted in the greatest phenol yield, further optimization of CFPS yield, particularly from multiple templates, could enable further increases in productivity.

### Example 5: Lysate Proteome Engineering Enables High Yield Ethanol Production in Crude Cell Extracts

Lysate-based cell-free systems provide a potentially economically viable opportunity to move chemical manufacturing away from live cells. These platforms could therefore be used to simplify and expedite the engineering of biomanufacturing processes. However, the efficiencies of lysates to convert simple sugars to more valuable products must be improved by shedding some of their biological complexity.

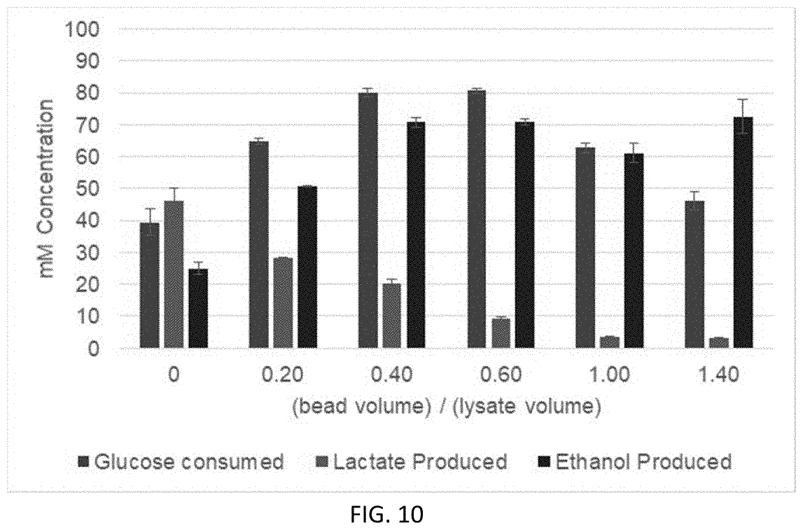

The inventors generated a 6×His-2 strain endogenously expressing 6×His-tagged LdhA and PflB proteins. A lysate derived from this strain can be treated with 6×His-tag binding cobalt beads to selectively reduce concentrations of LdhA and PflB from the lysate. Specifically, an extract derived from the 6×His-2 strain was incubated with cobalt beads at 0.2× the volume of lysate to allow binding of the beads to the two tagged proteins. The inventors found that the affinity-based manipulated lysate proteome can support the cell-free pooling of over 40 times more ethanol from glucose compared to control lysates. Assuming a black-box model, the amount of ethanol (EtOH) produced from consumed glucose (Glc) achieved by this lysate was approximately 32% of the maximum theoretical ethanol yield from glucose (0.51 g_{EtOH}/g_{Glc}). Ethanol accumulation was likely improved in these engineered lysates due to the activation of the ethanol synthesis pathway as an alternative cofactor regenerating module when LdhA and PflB concentrations are reduced. The inventors show here that the depletion method can be further optimized by increasing the bead-to-lysate volume ratio, suggesting more efficient pull-down of the tagged proteins. FIG. 10 shows that higher bead-to-lysate volume ratios lead to decreased flux towards lactate, the product of LdhA, and increased ethanol production. The lysate treated with a bead volume of 1.4× the lysate volume synthesized ethanol from consumed glucose at 78% of the maximum theoretical yield. This value is already the highest reported ethanol yield in cell-free systems to date. These results support that yields in cell-free systems can be significantly enhanced by selectively reducing the lysate proteome through the approach described herein.

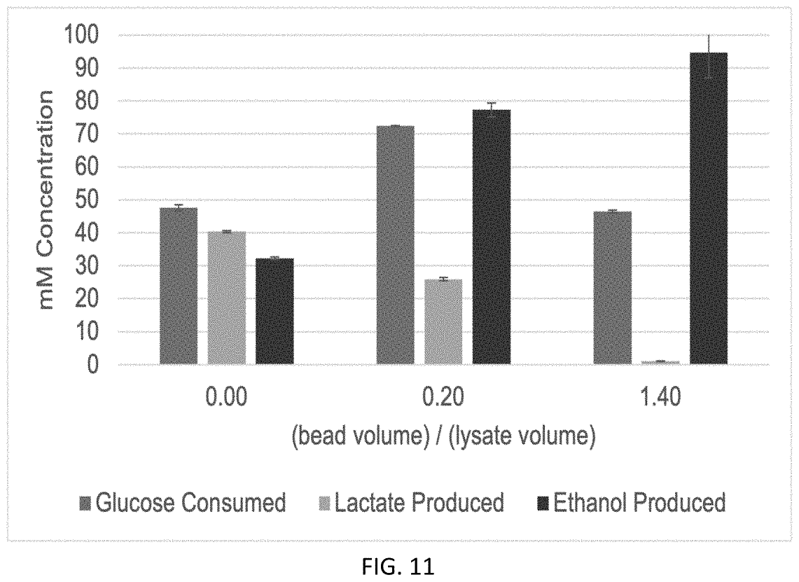

The inventors also have separately reported another lysate proteome engineering strategy which involves optimizing source strain cultivation conditions to enable the enrichment of target endogenous metabolic pathways in derived lysates. The inventors hypothesized that a combination of this approach with the improved depletion method described herein would allow higher yield ethanol synthesis. The inventors thus derived lysates from source strains grown in different percentages of carbon substrate and harvested at varying growth phases. These lysates were tested for their potential to convert glucose to ethanol at high yields. Specifically, source strains were first grown in 2×YPT media with 0.45%, 0.9%, 1.8%, 2.7%, and 3.6% glucose and harvested at OD₆₀₀ 6.0. Lysates derived from strains grown in 0.9% glucose had the highest ethanol yield (48%). Harvesting time was optimized by measuring ethanol yield in lysates derived from strains grown in 0.9% glucose to OD₆₀₀ 3.0, 4.0, 5.0, 6.0, and 7.0. Only lysates from strains grown to OD₆₀₀ performed with an ethanol yield above 50%. The inventors found that applying the aforementioned improved depletion method to a lysate prepared with optimized cultivation conditions can achieve 0.52 g_{EtOH}/g_{Glc}, corresponding to 102% of the maximum theoretical g_{EtOH}/g_{Glc} yield (FIG. 11). These results suggest that the pre-lysis (i.e., source strain cultivation) and post-lysis (i.e., selective removal of proteins) lysate proteome engineering methods are complementary and can be combined to achieve maximal yields in lysate-based cell-free systems.

1.-36. (canceled)

37. A cell-free extract that has a directed metabolic flux towards a metabolite of interest, comprising an extract from a genetically engineered cell, wherein at least one enzyme that affects the amount of the metabolite has been substantially removed from the cell extract.

38. The cell-free extract of claim 37, wherein multiple or all enzymes that affect the amount of the specific metabolite have been substantially removed from the cell extract.

39. The cell-free extract of claim 37, wherein the at least one enzyme is a central metabolism enzyme and deletion or inactivation of the at least one enzyme significantly impairs the cell's metabolism or kills the cell.

40. The cell-free extract of claim 37, wherein the genetically engineered cell further comprises a nucleic acid encoding an exogenous enzyme that affects the concentration of the metabolite.

41. The cell-free extract of claim 40, wherein the exogenous enzyme is selected from an enzyme not native to the cell or an engineered version of a native enzyme.

42. The cell-free extract of claim 37, wherein the at least one enzyme is selected from an enzyme in the TCA cycle, an enzyme in the Shikimate pathway, an enzyme in the pentose phosphate pathway, an enzyme in the 2-C-Methyl-D-erythritol 4-phosphate (MEP) pathway, an enzyme in the amino acid metabolism pathway, or an enzyme in the fatty acid metabolism pathway.

43. The cell-free extract of claim 37, wherein the metabolite is selected from a metabolite in the glycolysis pathway, a metabolite in the TCA cycle, a metabolite in the Shikimate pathway, a metabolite in the pentose phosphate pathway, a metabolite in the 2-C-Methyl-D-erythritol 4-phosphate (MEP) pathway, a metabolite in the amino acid metabolism pathway, or a metabolite in the fatty acid metabolism pathway.

44. The cell-free extract of claim 43, wherein the metabolite is selected from pyruvate, ethanol, mevalonate, isopentyl pyrophosphate, or acetyl coenzyme A.

45. The cell-free extract of claim 44, wherein the metabolite is isopentyl pyrophosphate, and wherein the enzyme is selected from geranyl pyrophosphate synthase, farnesyl pyrophosphate synthase, geranylgeranyl pyrophosphate synthase, or prenyl transferase.

46. The cell-free extract of claim 44, wherein the metabolite is acetyl coenzyme A, and wherein the enzyme is pyruvate dehydrogenase.

47. The cell-free extract of claim 37, wherein the genetically engineered cell has been engineered such that the at least one enzyme is linked to an affinity tag.

48. The cell-free extract of claim 47, wherein the affinity tag is selected from a His tag, a FLAG tag, a Strep II tag, a glutathione S-transferase (GST) tag, a Calmodulin binding protein (CBP) tag, a covalent yet dissociable NorpD peptide (CYD) tag, a polyarginine (Poly-Arg or nArg) tag, or a heavy chain of protein C (HPC) tag.

49. The cell-free extract of claim 37, wherein the genetically engineered cell has been cultured in a controlled growth medium before extract preparation.

50. The cell-free extract of claim 49, wherein the controlled growth medium lacks aromatic amino acids or comprises an organic hydrocarbon.

51. The cell-free extract of claim 49, wherein the controlled growth medium comprises a pre-defined temperature, pH, or oxygenation level.

52. The cell-free extract of claim 37, wherein the genetically engineered cell is a eukaryotic cell, a prokaryotic cell, or an archaeal cell.

53. The cell-free extract of claim 37, wherein the genetically engineered cell is a single-cell organism.

54. The cell-free extract of claim 37, wherein the single-cell organism is selected from the genera Lactobacillus, Escherichia, Bacillus, Vibrio, Bifidobacterium, Saccharomyces, Pichia, Pseudomonas, Streptomyces, or Streptococcus.

55. The cell-free extract of claim 37, wherein the genetically engineered cell is a bacterium from genus Escherichia, the metabolite is pyruvate, and the at least one enzyme is selected from PpsA, PflB, AceE or LdhA.

56. The cell-free extract of claim 55, wherein each of PpsA, PflB, AceE and LdhA is linked to the same affinity tag.

57.-76. (canceled)


SEQUENCE LISTING (12 sequences)

SEQ ID NO: 1 - DNA, 90 bp, Artificial Sequence
  Oligonucleotide
aataaaaaatccacttaagaaggtaggtgttacatgcaccatcaccatcaccattccgagcttaatgaaaagttagccacagcctgggaa

SEQ ID NO: 2 - DNA, 90 bp, Artificial Sequence
  Oligonucleotide
taaatgtgattcaacatcactggagaaagtcttatgcaccatcaccatcaccataaactcgccgtttatagcacaaaacagtacgacaag

SEQ ID NO: 3 - DNA, 90 bp, Artificial Sequence
  Oligonucleotide
caaaccgttcatttatcacaaaaggattgttcgatgcaccatcaccatcaccattccaacaatggctcgtcaccgctggtgctttggtat

SEQ ID NO: 4 - DNA, 90 bp, Artificial Sequence
  Oligonucleotide
actcaacgttattagatagataaggaataacccatgcaccatcaccatcaccattcagaacgtttcccaaatgacgtggatccgatcgaa

SEQ ID NO: 5 - DNA, 28 bp, Artificial Sequence
  Oligonucleotide
gccagccaggaaggactcgtcaccctcg

SEQ ID NO: 6 - DNA, 43 bp, Artificial Sequence
  Oligonucleotide
gcagtaaataaaaaatccacttaagaaggtaggtgttacatgc

SEQ ID NO: 7 - DNA, 24 bp, Artificial Sequence
  Oligonucleotide
cagcgtcatcatcataccgatggc

SEQ ID NO: 8 - DNA, 39 bp, Artificial Sequence
  Oligonucleotide
cttaaatgtgattcaacatcactggagaaagtcttatgc

SEQ ID NO: 9 - DNA, 23 bp, Artificial Sequence
  Oligonucleotide
gctggtttacgccgctttggtcc

SEQ ID NO: 10 - DNA, 34 bp, Artificial Sequence
  Oligonucleotide
accgttcatttatcacaaaaggattgttcgatgc

SEQ ID NO: 11 - DNA, 31 bp, Artificial Sequence
  Oligonucleotide
tggcctttatcgaagaaattttgctcgacag

SEQ ID NO: 12 - DNA, 28 bp, Artificial Sequence
  Oligonucleotide
atccacgtcatttgggaaacgttctgaa